(19) United States
(12) Patent Application Publication
(10) Pub. No.: US 2009/0012538 A1
(43) Pub. Date: Jan. 8, 2009

(54) METHODS AND DEVICES FOR CONTINUOUS SUTURE PASSING

(76) Inventors: Justin Saliman, Beverly Hills, CA (US); Erik Shahoian, San Ramon, CA (US)

Correspondence Address: SHAY GLENN LLP, 2755 CAMPUS DRIVE, SUITE 210, SAN MATEO, CA, 94403, US

(21) Appl. No.: 11/773,388

(22) Filed: Jul. 3, 2007

(51) Int. Cl. A61B 17/04

(52) U.S. Cl. 606/145; 606/148; 606/228

(57) ABSTRACT

Described herein are suture passers for suturing tissue in a continuous manner by passing a suture attached to a suture shuttle through. A suture passer may include a first jaw, a second jaw, and a tissue perpetrator that is retractable and extendable from the first jaw. The tissue penetrator may have a suture shuttle engagement region, and the second jaw may include a shuttle dock. The suture shuttle may be transferred between the first and second jaws as the tissue penetrator is extended from the first jaw and engages the second jaw. In some variations of the tissue passer, one or both jaws are tissue penetrating. In some variations, the jaws open in parallel, allowing large tissue regions to be positioned between the jaws. Methods of using these devices are also described, as are systems and kits including these devices.

### CROSS-REFERENCE TO RELATED APPLICATIONS

None

### BACKGROUND OF THE INVENTION

This invention relates to surgical stitching devices by which a stitch or continuous stitches may be made during surgery.

Suturing instruments for assisting a medical practitioner in placing stitches during surgical procedures are useful, particularly in surgical procedures requiring the placement of secure and accurate sutures in difficult to access regions of the body, including internal body regions. Instruments and methods for suturing remotely are especially important in minimally invasive surgical procedures such as laproscopic and endoscopic procedures. In addition to helping to access remote regions of the body requiring suturing, suturing instruments may also allow the efficient manipulation of very small needles and the formation of small and precise sutures

Arthroscopic rotator cuff repair is one example of a technically challenging procedure that requires the placement of sutures in difficult to reach regions, as well requiring precise placement of sutures. The procedure may be performed with the patient under general anesthesia, and small (e.g., 5 mm) incisions may be created in the back, side, and front of the shoulder, and an arthroscope and instruments may be switched between each of these positions as necessary. The rotator cuff tear may be visualized, and the size and pattern of the tear is assessed. Thin or fragmented portions are removed and the area where the tendon will be reattached to the bone is lightly debrided to encourage new blood vessel ingrowth for healing. Sutures may be placed to close a tear. Depending on the size and location of the tear, multiple suture stitches may be required. In many situations, an arthroscopic stitch passer and grasper are used to pass a suture through the tendon. A stitch passer and grabber are typically only capable of making a single stitch, and must be withdrawn and reloaded in order to make multiple stitches. Similarly, a separate arthroscopic knot tying instrument is typically used to pass and tie knots in the suture to secure the repair. Furthermore, most currently available suturing instruments are limited in their ability to be maneuvered, particularly over thicker tissue regions.

For example, the ArthroSew™ is a bi-directional suturing device with multiple-pass capability that has two jaws hinged to open V-like (from a common pivot). A suture is attached to the center of a double-ended needle and can be passed between the two jaws. At least one end of the needle protrudes from one or the other jaw at all times. The protruding needle may become caught in tissue, a problem that is exacerbated in difficult to access regions and regions offering limited maneuverability, such as the subacromial space of the jaw. Similar devices are described in U.S. Pat. No. 5,814,054, U.S. Pat. No. 5,645,552, U.S. Pat. No. 5,389,103, U.S. Pat. No. 5,645,552, and U.S. Pat. No. 5,571,090.

Other continuous suture passers include rotating suture passers, in which a curved suture needle is driven about an axis through successive revolutions to pass through an adjacent tissue, forming a spiral stitch through the tissue. U.S. Pat. No. 5,540,705 to Meade et al., describes one such embodiment.

### SUMMARY OF THE INVENTION

The devices described herein include continuous suture passers. Continuous suture passers are capable of passing a suture through a tissue multiple times without having to remove and reload the device. Thus, a continuous suture passing device is typically able to pass and retrieve a suture through a tissue. Any of the devices described herein may be used for continuous stitching and/or knot tying.

Described herein are continuous suture passers that include a first jaw and a second jaw, a handle including a jaw control to manipulate at least one of the jaws, an tissue penetrator configured to form a channel through the tissue through which a suture shuttle may be passed, and a suture shuttle dock functionally connected to the second jaw and configured to releasably secure the suture shuttle so that the suture shuttle may be passed between the first and second jaws. The tissue penetrator may be configured to be substantially retractable into the first jaw. For example, the distal tip of the tissue penetrator may be retracted into the first jaw.

In operation, a suture is passed from the first jaw to the second jaw and/or back from the second jaw to the first jaw. This may be accomplished using an extendable tissue penetrator that is connected to the first jaw. The extendable tissue penetrator can pierce the tissue, and can also engage a suture shuttle (to which a suture is attached) and thereby pull the suture shuttle through the passage that the tissue penetrator forms in the tissue. Extending the tissue penetrator forms a passage through the tissue, and can also pass the suture between the first and second jaws. For example, the tissue penetrator may include a suture shuttle engagement region (e.g., in a cavity within the tissue penetrator, along the outside of the tissue penetrator, etc.) to which the suture shuttle can be releasably attached. The suture can be passed from the tissue penetrator in the first jaw to or from a suture shuttle dock connected to the second jaw. Thus, both the tissue penetrator and the suture shuttle dock are configured to releasably secure the suture (usually attached to a suture shuttle). In some variations, the suture passer may pass a suture that is not attached to a suture shuttle. For example, the suture may be knotted, and the knot may be removably held by the jaws of the device (e.g., the tissue penetrator and a suture dock region). In some variations, the suture by itself is removably held by the suture passer and the dock region. The dock region may be similar to the shuttle dock region, and may include graspers for grasping the suture.

The suture passers described herein may also include a second tissue penetrator configured to be substantially retractable into the second jaw. The suture shuttle dock may be incorporated as part of this second tissue penetrator. In general, a tissue penetrator may incorporate a suture shuttle dock to which the suture shuttle (and connected suture) may be releasably secured. When the suture shuttle dock is incorporated into a tissue penetrator it may be referred to as a suture shuttle engagement region.

A suture shuttle dock or suture shuttle engagement region typically releasably secures a suture shuttle (and/or suture). A lock, latch, or other securement may be used to secure the suture shuttle to the suture shuttle dock so that the suture shuttle does not become inadvertently released. The attachment and release of the suture shuttle by the suture shuttle dock or suture shuttle engagement region may be regulated so that the suture is passed between the jaws only when the tissue penetrator is engaged with the suture shuttle dock. Thus, the device may be configured so that the tissue penetrator and the suture shuttle dock interface. For example, the suture shuttle dock may be configured to interface with the distal tip of the tissue penetrator, and the tissue penetrator may project at least partially into the suture shuttle dock to trigger transfer of the suture shuttle from or to the tissue penetrator or the suture shuttle dock. In some variations, the second jaw includes a channel through which the tissue penetrator from the first jaw may extend. Thus, the arrangement of the tissue penetrator connected to the first jaw and the suture shuttle dock connected to the second jaw may be coordinated so that they can correctly interact to transfer the suture shuttle. For example, the suture passer may be configured so that the relative positions of the tissue penetrator and the suture shuttle dock are substantially the same regardless of the position (closed, opening or opened) of the jaws. This is described more fully below.

In variations in which the tissue penetrator includes a shuttle engagement region, the shuttle engagement region may include a friction fit, a magnetic fit, a snap fit, a pressure fit, or some combination thereof. For example, the shuttle engagement region may be a channel within the tissue penetrator into which the suture shuttle may pass. The channel may be sized so that the walls of the channel engage with the outer diameter of the suture shuttle. Thus, the suture shuttle (and therefore the attached suture) may be secured within the tissue penetrator by friction between the suture shuttle and the channel. In some variations the shuttle engagement region is located on the outer surface of the tissue penetrator and the suture shuttle mates with the outer surface of the tissue penetrator. For example, the suture shuttle is a cuff or ring that slides over a portion of the tissue penetrator. As mentioned, a lock or stop may also be included as part of the shuttle engagement region (or the suture shuttle dock) to prevent the suture shuttle from disengaging until the suture shuttle is to be passed or cut.

The first jaw may include a first tissue contacting surface and the second jaw comprises a second tissue contacting surface, and further wherein the first tissue contacting surface and the second tissue contacting surface are substantially parallel when the jaws are open. Parallel opening of the jaws may allow larger thickness or areas of tissue to be grasped or manipulated (e.g. sutured), because as the jaws close they apply approximately the same force over a large area of the tissue, rather than over a small region. This may prevent “pinching” or damage to tissue, particularly near the region in which the jaws are hinged. Furthermore, the suture passer jaws may also act as a tissue clamp or grasper, holding the tissue securely as the suture is passed through it.

The suture passers described herein may be described as having proximal and distal regions (as well as intermediate regions therebetween). For example, the first and second jaws may comprise the distal end of the device, and the handle may be in the proximal region of the device. The region between the proximal and distal regions may be an elongate neck region. In some variations, the suture passer is configured for arthroscopic (or endoscopic) use by keeping the dimensions of the device (particularly the distal and neck regions) small enough so that they are compatible with arthroscopic or endoscopic methods and devices. For example, the diameter of the distal end of the device may be less than about 15 mm when the device is closed. In some variations, the diameter of the distal end of the device is less than 12 mm, less than 10 mm, less than 8 mm, or less than 6 mm. Similarly, the jaws at the distal end of the device may be appropriately scaled. For example, the first and second jaws may be less than about 30 mm long, less than 21 mm long, less than 25 mm, less than 20 mm long, etc. The elongate neck region may have a diameter that is less than the diameter of the closed first and second jaws. The jaws in the distal region are typically oriented so that they open and close perpendicular to the long axis of the device, however alternative configurations are possible.

In general, the handle of the device is configured to be grasped (e.g., by a doctor) at the proximal end of the device and to control the operation of the distal end of the device. Thus, the handle may be configured to conform to a person's hand and may include one or more controls for controlling the operation of the device, including the opening/closing of the jaws and the extension/retraction of the tissue penetrator, and the passing of the suture between the jaws. For example, the handle may include a jaw control that can be used to manipulate the jaws. As described more fully below, the jaws may open and close by moving one jaw relative to the other jaw, or by moving both jaws relative to each other. Thus, the jaw control may control the opening of the jaws by controlling movement of one jaw (e.g., the first jaw) or both jaws.

Any appropriate control may be used as the jaw control. For example, the jaw control may be a trigger, a button, a dial, slider, etc. The jaw control may be a proportional control, so that movement of the control (e.g., squeezing a trigger, turning a dial, etc.) results in a proportional movement of the jaw(s). The proportion may be 1:1, or some other ratio (and may be adjustable in some variations). In some variations, the jaw control includes a lock so that the jaws may be held in the selected position when the jaw control lock is engaged. Locking may be particularly useful when tying knots and when passing the suture between the jaws.

The jaw control may operate by any appropriate principle. For example, the jaw control may include a plurality of coaxial members that control jaw opening and closing. Thus, in some variations a wire or pusher may be connected to a trigger of the jaw control, and the wire or pusher may be connected to a jaw or to both jaws to open and close the jaws. These pusher or wire members may be coaxial, so that the wire or wires are contained within the elongated neck region of the device between the proximal and distal ends. In some variations, the control includes an electrical control that activates a motor to move the jaw(s). In some variations, the control includes a magnetic control that magnetically moves the jaw(s), a pneumatic control that pneumatically moves the jaw(s), a hydraulic control that hydraulically moves the jaws, or the like (including combinations thereof).

A jaw control may be a proportional control that is limited. Thus, a limiter may be included to limit the motion of the jaw(s) when the jaw control is used. A limiter may prevent the jaws from applying pressure above some threshold, preventing or limiting damage to the tissue, while still allowing it to be secured between the jaws. In some variations, this limiter is adjustable. The limiter may not completely prevent additional pressure from being applied, but it may limit it by, for example, increasing the proportionality of the proportional jaw control. Thus, as more force is applied to the control, smaller and smaller percentages of this force are translated to the tissue (either linearly or above a threshold). The force applied may be fed back to the limiter along the control mechanism. For example, the limiter may be in communication with the mechanical control mechanism (e.g., cables, pushers, etc.), pneumatic control mechanism, magnetic control mechanism, electric control mechanism, etc. In some variations, the limiter limits force applied by the jaws when the force exceeds about 20 lb (or about 5 lb, about 10 lb, about 15 lb, about 25 lbs, etc.). The linkage between the jaw movement and the jaw control may include a mechanical advantage (e.g., approximately 1.25:1). Thus, if you apply 1 pound of force to the jaw control, the jaw exerts 1.25 pounds of clamping force on the tissue over a range of pressures (particularly in variations including a limiter).

In some variations, the handle of a tissue penetrator also includes a tissue penetrator control configured to extend the tissue penetrator from the retracted position (e.g., at least partially within the first jaw) to an extended position, wherein tissue between the jaws is pierced by the tissue penetrator. Thus, the handle may include a separate tissue penetrator control configured as a trigger, dial, button, slider, etc. In some variations, the tissue penetrator control is a proportional control so that movement of the control results in proportional movement of the tissue penetrator. Thus, the tissue penetrator may be extended by applying pressure to the tissue penetrator control (to pierce tissue) and retracted by decreasing the amount of force applied. For example, the control may be biased (e.g., by a spring) so that return of the control to the relaxed position causes return of the tissue penetrator to the retracted position. In some variations, the tissue penetrator is triggered and fully extends after triggering, and returns automatically to the retracted position.

In some variations, the tissue penetrator is controlled by the jaw control. For example, when the jaw control is activated to close tissue between the jaws, the tissue penetrator is activated. In some variations the tissue penetrator is extended when the pressure applied to the jaw control exceeds some threshold. For example, the tissue penetrator may be extended when the force applied to the jaw control exceeds about 2 lbs, about 5 lb, about 8 lb, about 10 lb, about 12 lb, about 15 lb, about 20 lb, etc. In some variations, the tissue penetrator movement is linked to additional pressure on the jaw control, so that continuing to squeeze the jaw control further extends tissue penetrator. In some variations, the tissue penetrator is “triggered” and fully extends after triggering, regardless of the amount of pressure applied to the jaw control above the threshold to trigger. Return of the tissue penetrator from the extended (tissue piercing) configuration to the contracted (at least partly within the jaw) configuration may be automatic or controlled (e.g., by decreasing the pressure on the jaw control).

In addition to the jaw control, the handle of a suture passer may include additional controls that can control other aspects of the device, such as the angle of deflection of the distal end of the device. Thus, in some variations the distal end of the device (e.g., including the jaws or a portion of the jaws) may be bent or deflected. Deflection of the distal end may help improving steering of the device within a subject's body, as well as improving access to difficult to reach regions. In some variations, the handle includes a distal tip deflector control that is configured to control deflection of the distal end of the device.

The suture passer devices described herein may also be configured so that they are compatible with surgical visualization techniques. For example, the suture passer may include one or more markers that can be visualized (e.g., radio-opaque markers, electrodense markers, fluorescent markers, etc.). In some variations, the first and second jaws are made of a substantially transparent material so that the tissue may be visualized through them.

Because the suture may be passed back and forth between the jaws of the devices described herein, the device may be adapted so that the suture does not interfere with the operation of the device as it is passed. For example, the suture passer may include one or more guides or channels into which the suture may reside when the suture shuttle is held by the first jaw (e.g., by the suture passer of the first jaw) or the second jaw (e.g., by the suture shuttle dock). Thus, the first jaw and/or the second jaw may include a suture passage to at least partially guide the suture. In particular, the tissue penetrator and/or the suture shuttle dock may include a suture passage to at least partially guide the suture when the suture shuttle is engaged with the tissue penetrator. The suture passage (which may be a channel or guide, and may therefore be referred to as a suture channel or suture guide) may be open (e.g., an open channel) or at least partially enclosed.

Also described herein are continuous suture passers including a tissue clamping region having two jaws, a handle including a jaw control to manipulate at least one jaw, an elongate neck region between the distally located tissue clamping region and the proximally located handle, and a tissue penetrator configured to form a channel through the tissue through which a suture shuttle may be passed. The tissue penetrator may be configured to be substantially retractable into the first jaw (e.g., the distal tip of the tissue penetrator may be retracted into the first jaw). The tissue clamping region typically includes a first jaw having first tissue-contacting surface, and a second jaw having a second tissue-contacting surface, wherein the tissue-contacting surfaces of the first and second jaws are substantially parallel when the tissue clamping region is opened. The device may also include a suture shuttle dock functionally connected to the second jaw and configured to releasably secure the suture shuttle. Any of the features previously described (alone or in combination) may be included as part of these continuous suture passers.

Also described herein are continuous suture passers that include a first jaw and a second jaw, a handle including a jaw control to manipulate at least one jaw, a first tissue penetrator configured to extend from the first jaw and to form a channel through the tissue through which a suture shuttle may be passed (wherein the suture shuttle may be releasably secured to the first tissue penetrator), a second tissue penetrator configured to extend from the second jaw and to form a channel through the tissue which the suture shuttle may be passed (wherein the suture shuttle may be releasably secured to the second tissue penetrator), and the first and second tissue penetrators are configured to pass the suture shuttle between them. Thus, the tissue penetrators may meet in the tissue and the shuttle (with suture) can be passed between them.

Also described herein are continuous suture passers having suture channels, passages or guides for controlling the position of the suture with respect to the distal and/or intermediate region of the suture passer as the suture passer is operated. For example, a suture passer may include a first jaw and a second jaw, a handle including a jaw control to manipulate at least one jaw, and a first suture shuttle dock functionally connected to the first jaw, wherein the first suture shuttle dock is configured to releasably secure a suture shuttle so that the suture shuttle may be passed between the first and second jaws. The first suture shuttle dock may include a suture passage to at least partially guide a suture connected to the suture shuttle when the suture shuttle is engaged with the first suture shuttle dock. The suture passage (or guide) may allow the shuttle to reside completely within the jaws, and because it prevents the suture from interfering with the penetrator as the tissue penetrator passes through tissue or interacts with the opposite jaw (e.g., a shuttle dock region on the opposite jaw).

In some variations, the suture passer may also include a second suture shuttle dock functionally connected to the second jaw. The second suture shuttle dock may include a second suture passage to at least partially guide a suture connected to the suture shuttle when the suture shuttle is engaged with the second suture shuttle dock. The suture passages may be openings or channels through the first and second jaws (e.g., in the region of the shuttle dock). For example, the suture passage may include a notch or opening in the shuttle dock through which the suture may be guided. In some variations, the suture passage may have a substantially smooth surface against which the suture may rest. For example, the suture passage may be coated, polished or buffed.

In some variations, the suture passer also includes an intermediate region between the first and second jaws and the handle that has a suture channel that is configured to guide the suture extending from the first or second jaws. For example, the suture channel may be an opening extending longitudinally along the intermediate region. Thus, the suture channel may prevent the suture from impinging between the shaft of the device an inner wall of a cannula which may be used to position the suture passer, particularly in endoscopic and arthroscopic applications.

Also described herein are kits including a suture passer as described herein and instructions for use.

Also described herein are suture shuttles. A suture shuttle may include a suture attachment region configured to secure to a surgical suture, a shuttle body having an outer surface that is substantially blunt, and a shuttle retainer region configured to engage a tissue penetrator to retain the suture shuttle in communication with the tissue penetrator. The shuttle body region may have an outer diameter that is less than 5 mm. The suture shuttles described herein may also include a shuttle alignment feature, wherein the shuttle alignment feature communicates with a region of the tissue penetrator and a region of a suture shuttle dock to orient the suture shuttle. A shuttle alignment feature may help organize and control the suture attached to the suture shuttle. For example, by helping position the suture into a suture guide or channel as the suture shuttle is passed between the jaws of a suture passer. A shuttle alignment feature may include a groove in the shuttle body.

In some variations, the shuttle body region is substantially spherical. In some variations, the suture shuttle body is substantially ellipsoid, or substantially elongate. Virtually any shape (particularly blunt shapes) may be used. In variations of the suture shuttle to be secured within the tissue penetrator, the suture shuttle should be small enough to mate with the suture shuttle dock and the shuttle engagement region of the tissue penetrator.

The suture attachment region generally acts as the attachment region for the suture. In some variations, the suture attachment region of the suture shuttle comprises a loop, a hook, a passage, or a channel. A suture may be passed through the suture attachment region, and may be tied, glued, welded, or otherwise affixed to the suture attachment region. In some variations the suture may be attached or connected to the suture attachment region “on the fly”, so that a suture that has already been passed through the tissue may be connected to the suture shuttle and used with a suture passer as described herein.

The suture shuttle body region may be made of a polymer, a metal, or a ceramic. For example, in some variations, the material that comprises the outer surface of the suture shuttle helps secure the suture shuttle to the suture passer (e.g., to the tissue penetrator or the suture shuttle dock). Thus, a portion of the outer surface of the shuttle body may be configured as a shuttle retainer region. Any of the suture shuttles described herein may include a suture attached thereto.

Also described herein are systems for continuously suturing a tissue. The system may include a continuous suture passer and a suture shuttle. The continuous suture passer may have a first jaw and a second jaw, a handle including a jaw control configured to manipulate at least one jaw, and an extendable tissue penetrator configured to form a channel through the tissue (wherein the tissue penetrator is configured to be substantially retracted into the first jaw). The suture shuttle may include a suture attachment region configured to secure to the distal portion of a surgical suture, and a shuttle body that is configured to engage the tissue penetrator. The system may also include a suture attached to the suture attachment region of the shuttle. Any of the suture passers described herein may be part of a system including a suture shuttle. For example, the suture passer may be a continuous suture passer that includes a suture shuttle dock functionally connected to the second jaw and configured to releasably secure the suture shuttle. The continuous suture passer used as part of the system may include a tissue clamping region including a first tissue-contacting surface on the first jaw and a second tissue-contacting surface on the second jaw, wherein the tissue-contacting surfaces of the first and second jaws are substantially parallel when the tissue clamping region is opened.

Also described herein are methods of passing a suture that include the steps of positioning a tissue between a first jaw and a second jaw of a suture passer (wherein the suture passer included an extendable tissue penetrator functionally connected with the first jaw and a suture shuttle dock functionally connected with the second jaw, and wherein a suture shuttle is releasably held by either the suture shuttle dock or the retractable tissue penetrator), extending the tissue penetrator through the tissue from a retracted position, transferring the suture shuttle between the suture shuttle dock and the retractable tissue penetrator, retracting the tissue penetrator through the tissue, repositioning the tissue between the first jaw and the second jaw of the suture passer, extending the tissue penetrator through the tissue, and transferring the suture shuttle between the suture shuttle dock and the retractable tissue penetrator. This method may also include the steps of securing the tissue between the first jaw and the second jaw before extending the tissue penetrator through the tissue.

A suture shuttle may be loaded into the suture passer before beginning this method (or as a first step). In some variations, the suture is first loaded into the suture passer.

In some variations, the first and second jaws are separated so that the first and second jaws are substantially parallel.

In partial operation, the suture shuttle may be transferred from the suture shuttle dock to the tissue penetrator by contacting the suture shuttle with the tissue penetrator, and releasing the suture shuttle from the suture shuttle dock. The suture shuttle may be transferred from the tissue penetrator to the suture shuttle dock by disengaging the suture shuttle from the extended tissue penetrator.

Also described herein are methods of passing a suture that include the steps of expanding a tissue clamping region of a suture passer (wherein the tissue clamping region has a first jaw with a first tissue-contacting surface and a second jaw with a second tissue-contacting surface) so that the first tissue-contacting surface and the second tissue-contacting surface are substantially parallel, positioning a tissue between the first and second jaws, clamping the tissue between the first and second jaws, extending a retractable tissue penetrator into the tissue from the first jaw after clamping the tissue between the first and second jaws, engaging a suture shuttle with the tissue penetrator, and retracting the tissue penetrator and pulling the suture shuttle through the tissue. In some variations, the tissue is clamped between the first and second jaws with a pressure between the first and second jaws that is less than a maximum pressure. For example, the tissue may be clamped between the jaws with a threshold pressure that is less than about 10 lb, less than about 12 lb, less than about 15 lb, less than about 20 lb, etc.

In some variations, the suture passer further comprises a handle having a jaw control, and the tissue is clamped between the first and second jaws of the suture passer by applying pressure to a jaw control on a handle. The jaw control may include a limiter that limits the amount of pressure applied by the first and second jaws against the tissue when pressure is applied to the jaw control. The retractable tissue penetrator may be extended when the pressure applied to the jaw control exceeds a predetermined amount.

The method may also include the steps of extending the retractable tissue penetrator and engaged suture shuttle through the tissue. The tissue penetrator may be extended through the tissue to contact at least a portion of the second jaw. The method may also include the step of unclamping the tissue front between the first and second jaws. Once the tissue has been unclamped, it may be repositioned between the first and second jaws, and then clamping the tissue between the first and second jaws again.

The methods of passing a suture described herein may be used to form virtually any number of suture stitches that require multiple passes of the suture through tissue. For example, a modified Mason-Allen stitch may be particularly useful for orthopedic and other applications and may be formed by the methods described herein, using the continuous suture passers. The methods described herein may be used as part of any appropriate medical procedure, including (but not limited to) arthroscopic and endoscopic procedures. For example, the methods described herein may be used as part of an orthopedic procedure, such as rotator cuff tendon repair, labral tissue repair, to capsular tissue repair, etc.

### INCORPORATION BY REFERENCE

All publications and patent applications mentioned in this specification are herein incorporated by reference in their entirety to the same extent as if each individual publication or patent application was specifically and individually indicated to be incorporated by reference.

### BRIEF DESCRIPTION OF THE DRAWINGS

FIG. 1 is one variation of a suture passer as described herein.

FIG. 2 is another variation of a suture passer.

FIG. 3 is another variation of a suture passer.

FIG. 4A is another variation of a suture passer.

FIGS. 4B and 4C show side perspective views of the distal end of the suture passer in FIG. 4A in open and closed configurations, respectively.

FIGS. 5A-5C illustrate the operation of the jaws of one variation of a suture passer as described herein.

FIG. 6A is one variation of the distal end of a suture passer.

FIG. 6B is a perspective view of the lower jaw of the suture passer of FIG. 6A, and FIGS. 6C-6G are side views of the front, back, side, top, and bottom (respectively) of the same lower jaw.

FIGS. 7A-7C illustrate the operation of a tissue penetrator coupled to a first jaw.

FIG. 8 shows a tissue penetrator retracted into the first jaw.

FIGS. 9A-9C are side, top, and front perspective views (respectively) of a tissue penetrator as described herein.

FIGS. 10A-10E illustrate the operation of one variation of a suture passer passing a suture through relatively thick tissue.

FIGS. 11A-11F illustrate the operation of one variation of a suture passer passing a suture through relatively thin tissue.

FIGS. 12A-12D illustrate the distal region of a suture passer.

FIG. 13A is another view of the distal end of a suture passer with a tissue penetrator extended.

FIG. 13B is a partially cut-away view of the distal end of a suture passer in which the tissue penetrator is engaged with the opposite jaw.

FIG. 13C is the view shown in FIG. 13B in which the lower jaw is also illustrated.

FIG. 14 is one variation of a suture shuttle.

FIG. 15A is another variation of a suture shuttle, and FIGS. 15B and 15C are perspective and side views of the suture shuttle as described herein.

FIG. 16 is another variation of a suture shuttle.

FIGS. 17A and 17B illustrate attachment of a suture to one variation of a suture shuttle.

FIGS. 18A and 18B illustrate operation of one variation of a suture shuttle.

FIG. 18C is another variation of a suture shuttle.

FIG. 18D is a tissue penetrator that may be used with the suture shuttle shown in FIG. 18D, and FIG. 18E is the tissue penetrator of FIG. 18D with a suture shuttle removably attached thereto.

FIGS. 19A-19C illustrate attachment of a suture to one variation of a suture shuttle.

FIG. 19C is another variations of a suture shuttle similar to the variation shown in FIGS. 19A-19C.

FIGS. 24A and 24B illustrate detail of one variation of a handle.

FIGS. 24C and 24D illustrate the conjugate motion of the handle to operate the jaws.

FIGS. 25A-25E illustrates one method of stitching using a continuous suture passer as described herein.

FIGS. 26A-26C illustrate the operation of one variation of the distal end of a continuous suture passer.

FIG. 27A is one variation of a continuous suture passer.

FIG. 27B is one variation of tissue penetrator.

FIG. 27C shows a suture passer similar to the device shown in FIG. 27A.

FIGS. 27D-27G illustrate the operation of suture passer as shown in FIG. 27A.

FIGS. 28A and 28B show side perspective views of the distal end of another variation of a suture passer.

FIG. 28C is a side view of the distal end of the suture passer shown in FIG. 28A

FIG. 28D is a side view of the proximal end of the suture passer shown in FIG. 28A.

FIGS. 29A-29B illustrate the operation of another variation of a suture passer.

FIGS. 30A and 30B show side and top perspective views of one variation of a suture shuttle.

FIG. 31 is a cross-section through one variation of a jaw of a suture passer.

FIG. 32 is a cross-section through another variation of a suture passer.

FIG. 33 is another cross-section through one variation of a suture passer.

FIGS. 34A-34C illustrate the operation of another variation of a suture passer.

FIGS. 35A and 35B illustrate the operation of another variation of a suture passer.

FIG. 36 is a cross-section through one variation of a jaw of a suture passer.

FIGS. 37A and 37B are side perspective and side views (respectively) of the distal end of another variation of a suture passer.

FIG. 38 is a guard or shield.

FIGS. 39A and 39B are schematic illustration of the shoulder region of a subject, including a rotator tendon region.

### DETAILED DESCRIPTION OF THE INVENTION

Described herein are suture passers for passing a suture through tissue, as well as systems including suture passers, and methods of passing sutures through tissue. In general, the suture passers described herein are continuous suture passers that are configured to pass a suture back and forth through a tissue without having to reload the device. Thus, these devices may be used for continuous stitching of tissue.

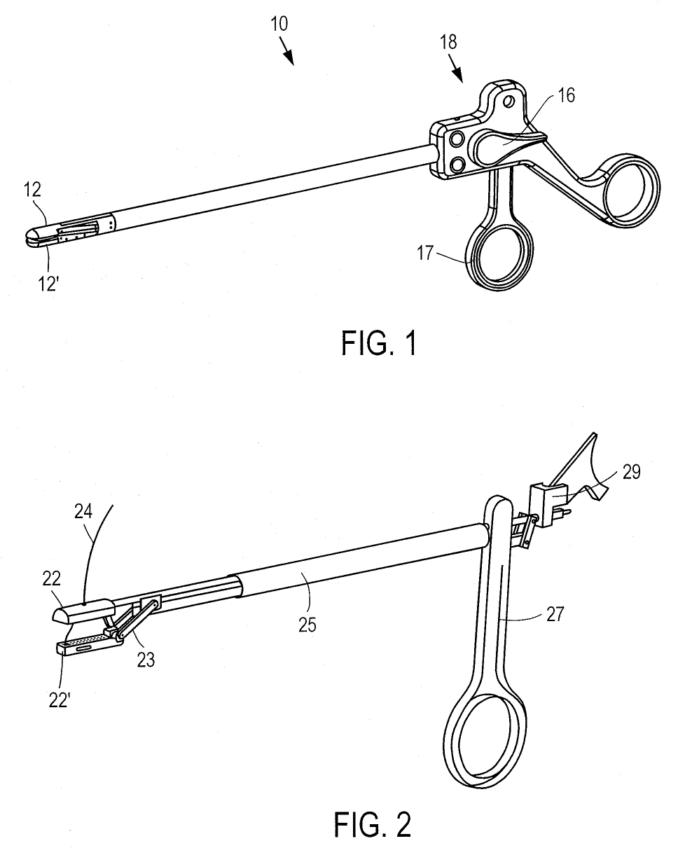

FIG. 1 illustrates one variation of a continuous suture passer 10, including some of features that may be included in a continuous suture passer. In general, the suture passers described herein may include a distal, tissue-contacting region and a proximal region having a handle or other user interface. The distal end of the device may be separated from the proximal end of the device by an elongate intermediate region. The intermediate region may be any appropriate length, and may be straight (as shown in FIG. 1) or it may be curved or otherwise shaped. In some variations, the intermediate region is flexible or bendable. The intermediate region may be a tubular shape, or it may have other cross-sectional profiles. The intermediate region may be hollow, and may enclose linkages between the distal end of the device and the proximal end of the device.

The distal end of the suture passer 10 shown in FIG. 1 has two jaws 12, 12′ that are shown in the closed position. The opening and closing of the jaws may be controlled by handle 18. This handle 18 is configured to be grasped and manipulated by a subject's hand. The handle 18 also includes a jaw control lever 17 that may be moved to open and close the jaws 12, 12′ of the device.

The handle shown in FIG. 1 also includes a tissue penetrator control 16, which is can manipulate a tissue penetrator and to pass the suture between the jaws, as described in more detail below. In this example, the tissue penetrator control 16 is a switch or lever. In general, the handle may include any appropriate control, one or more locks, including locks for locking the jaws, locking the tissue penetrator, locking the suture passer, etc. These controls may be switches, levers, buttons, sliders, dials, triggers, etc.

FIG. 2 shows another variation of a suture passer. In FIG. 2, the distal end of the suture passer has two jaws 22, 22′, which are shown in an open configuration. In this example, the upper jaw 22 remains in the plane of the intermediate section 25 while the lower jaw 22′ opens. The distal end of the device also includes a hinge region 23, along which the jaws are opened. A suture 24 is also included, and is shown passing through the upper jaw 22 while the distal end of the suture (connected to a suture shuttle) is held within the lower jaw 22′. The proximal end of the device includes a handle 27. The device also includes a jaw control 29 that may be used to open and close the jaws. This jaw control is a thumb-lever 29.

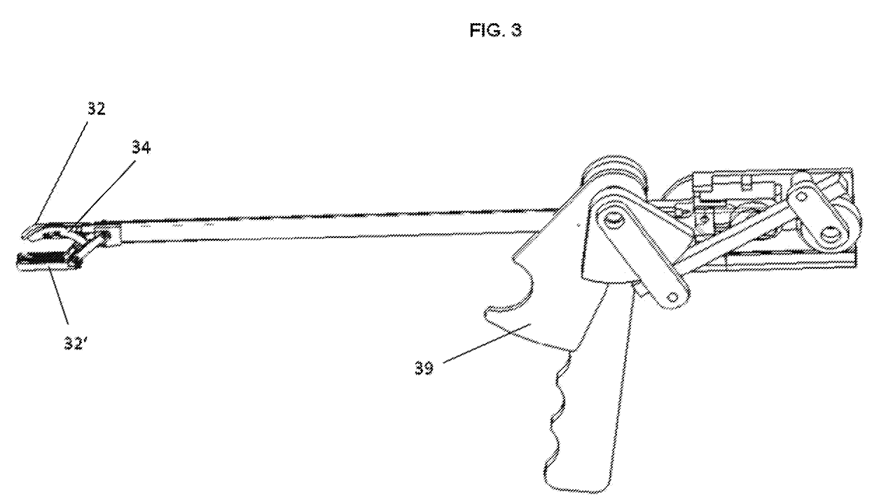

Another example of a continuous suture passer is shown in FIG. 3. In FIGS. 2 and 3, the handle region is shown in skeletonized view, meaning that components (including an outer cover), are not shown, but may be used as part of the handle. A cover may include additional grasping surface area for the user, and may also help protect the device, and particularly any moving components, which are shown as visible in FIG. 3. In FIG. 3 the distal end of the device includes two jaws 32, 32′ that are shown in partial cut-away view so that the tissue penetrator 34 is visible, retracted in the upper jaw. The relative motions of the upper and lower jaws 32, 32′ and the tissue penetrator are described in greater detail below. As described above, the jaw movement may be controlled by the jaw control 39.

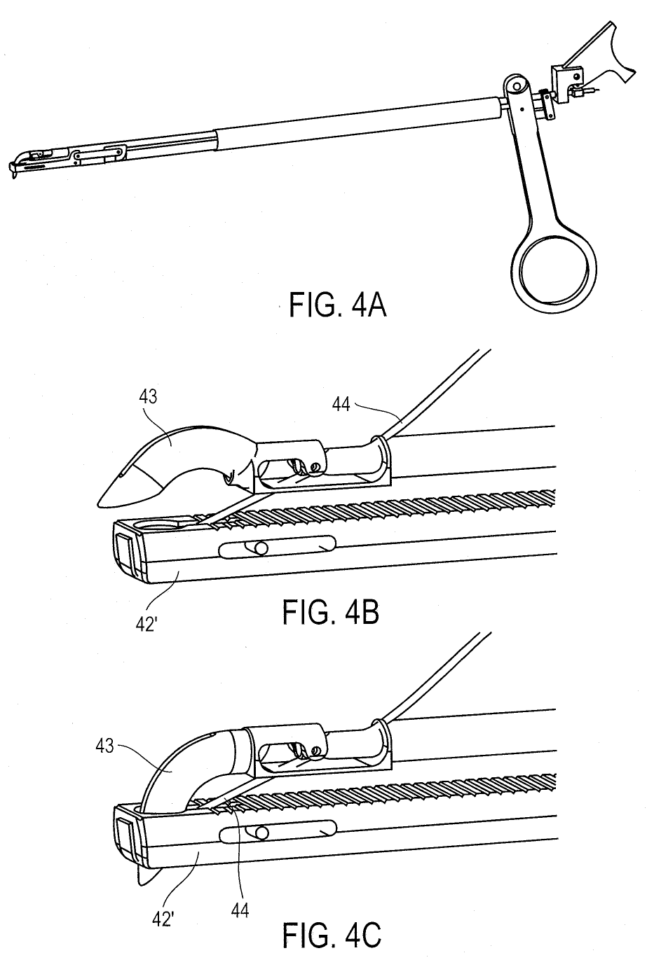

FIG. 4A shows another variation of a continuous suture passer whose proximal end is similar to the suture passer shown in FIG. 2. FIGS. 2B and 2C show the distal end of the device in more detail. In this variation, the lower jaw 42′ of the suture passer in FIG. 4 is also similar to the devices shown in FIGS. 1-3, however the upper jaw is configured as a tissue penetrator 43. This tissue penetrating upper jaw 43 may be opened (as shown in FIG. 4B) so that tissue may be placed between the upper jaw and the lower jaw 42′. A suture 44 is shown extending from the lower jaw 42′. In this example the tissue penetrator 43 is hinged so that it can open and close. When the upper, tissue penetrating jaw is closed as shown in FIG. 4C, the tissue penetrator passes through any tissue between the upper jaw and lower jaw, and the tissue penetrator passes at least partially through the lower jaw 42′ so that a suture shuttle (not visible in FIG. 4A-C) may be passed between the upper tissue penetrating jaw and the lower jaw.

FIGS. 1 through 4A give a general overview of a few variations of the continuous suture passers described herein. In general these, tissues passers include distal tissue engaging jaws, and a proximal handle including one or more controls for controlling the suture passer. The distal jaws are also configured to continuously pass a suture attached to a suture shuttle back and forth between the jaws and any intervening tissue. Various features and aspects of the suture passers are described in greater detail below, and exemplified in the figures. Although specific variations of the devices are shown having different features or components, any of the features or components described herein may be used in combination with any of the other suture passers or elements of suture passers described.

In general, the suture passers described herein may include a first jaw and a second jaw mat are arranged opposite each other so that tissue may be placed or held between them. A suture attached to a suture shuttle may be passed between the two jaws. Thus, the jaws may be adapted so that the suture shuttle may be releasably held by each jaw. One or both jaws may include a tissue-contacting surface. The inner surface of the jaws (the surface that faces the opposite jaw) may include a surface that is adapted to contact tissue. For example, the tissue-contacting surface may help grip tissue between the jaws by including a texture or material (coating, layer, etc.) that helps grasp or secure tissue. In some variations, the tissue-contacting surface includes ridges, bumps, or other irregularities to help grasp the tissue. In some variations, the tissue-contacting surface includes a compliant material (e.g., rubber, silicone, etc.) to prevent or minimize damage to the tissue as it is held between the jaws.

One or both of the jaws may open and close in any appropriate manner. For example, the jaws may be hinged so that one or both of the jaws open “V”-like, around a hinge point. For example, the jaws may be hinged (by a pivot or a living hinge) to form an opening therebetween. As mentioned above, the opening and closing of the jaws may be controlled by a jaw controller. In some variations, only one jaw (e.g., the first jaw) moves apart from the other (second) jaw to form an opening between the jaws. In some variations, both the upper and lower jaw move to open and close the jaws.

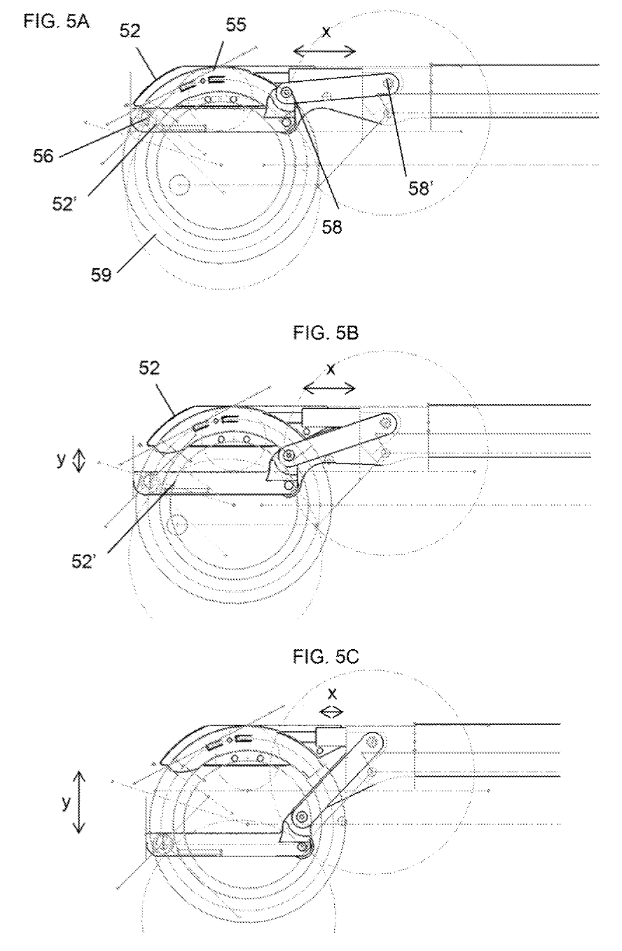

In some variations, the jaws open so that the upper and lower jaws remain substantially parallel. In some cases, this may mean that the tissue-contacting surfaces of the upper and lower jaws are substantially parallel. This is illustrated in FIGS. 5A-5C, which illustrate the motion of an upper and a lower jaw of a suture passer. In FIG. 5A the upper jaw 52 and the lower jaw 52′ are closed. The upper and lower jaws are shown in a transparent view in FIGS. 5A-5C, so that internal details may be seen. For example, a tissue penetrator 55 is visible and is at least partially retracted into the upper jaw 52. The lower jaw includes a suture shuttle dock 56 that is visible. The lower jaw is hinged 58, 58′ and configured so that it opens downward, as shown in FIG. 5B. The lower jaw 52′ remains substantially parallel to the upper jaw 52 as the jaws open. In FIG. 5C, the lower jaw has opened more fully. When the jaws open so that the tissue-contacting surfaces are substantially parallel, as shown in FIG. 5C, a relatively large and thick portion of tissue may be inserted between the jaws without putting pressure on the more proximal regions of the tissue within the jaws (the region nearer the hinge). This may prevent damage to the tissue. In addition, the jaws may be used as a clamp to secure the tissue, and the clamping jaws may provide a uniform pressure across the tissue.

In FIGS. 5A-5C the inner (tissue-contacting) surfaces of the jaws are shown as parallel over the length of the jaw, meaning the tissue contacting surface of the jaws are separated by approximately the same distance over the length of the jaws. In some variations, the jaws are substantially parallel even when the distance between the surfaces of the jaws varies slightly over the length of the jaws. For example, the jaws may be substantially parallel even when the distance between the jaws in the proximal end of the tissue contacting surface is less than about 5%, less than about 10%, less than about 20%, less than about 40%, the distance between the jaws in the distal end of the tissue contacting surface. Thus “substantially parallel” does not require that the jaws be strictly parallel.

The motion of the jaws as they open and close may be coordinated so that features of the upper and lower jaws remain in alignment as the jaws are opened or closed. The alignment of features between the upper and lower jaw may be achieved by configuring the first and/or second jaws so that they move with compound motion. Thus, one or both jaws may be configured for compound motion when opening and closing. Compound motion in this context may refer to combinations of simple motions (e.g., straight translation, rotation, helical, etc.). For example, as the tissue-contacting surface of the first jaw 52′ is moved away from the tissue-contacting surface of the second jaw 51 in FIGS. 5B and 5C, the second jaw 51 is also moved inwards (proximally). This is apparent in FIGS. 5A-5C by the difference in the lengths of x, x′ and x″, while the opening of the jaws is indicated by the transition in distance y′ to y″. In some variations, the same jaw moves both away from the other jaw (y) and proximally and distally (x).

The coordinated compound motion of the jaws may allow maintenance of the jaw alignment, so that even when the jaws are not fully closed (or opened) a tissue penetrator extending from one jaw may contact the suture shuttle dock on the other jaw, so that the suture shuttle can be reliably transferred between the jaws. This alignment may also be referred to as “kinematic linkage” between the first and second jaws. For example, the tissue penetrator 55 in FIGS. 5A to 5B is configured to move in a semi-circular arc, as indicated by the circular outlines 59. This outline 59 tracks the motion of the tissue penetrator as it would extend from the upper jaw 52. Because of the compound motion of the upper 52 and lower 52′ jaws, the distal tip of the tissue penetrator 55 and the suture shuttle dock 56 fall on the circular outline 59 as the jaws open and close (in all of FIGS. 5A-5C). In practice, this means that the tissue penetrator 55 will engage with the suture shuttle dock 56 if the tissue penetrator is extended from the upper jaw regardless of how opened or closed the jaws are.

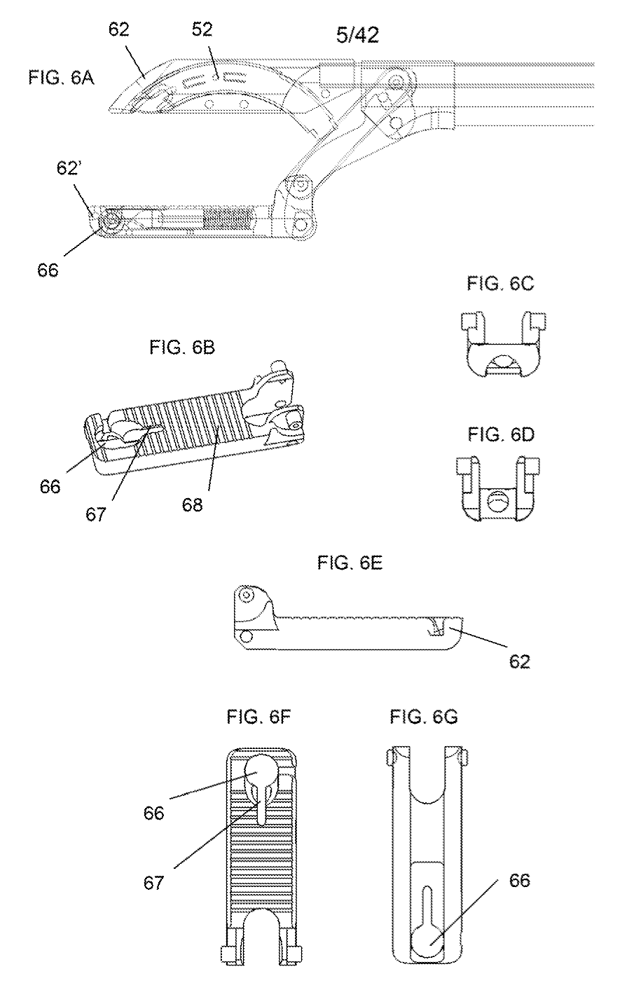

FIGS. 6A to 6G illustrate additional details of the first and second jaw shown in FIG. 5. FIG. 6A shows a line drawing of the first 62 and second 62′ jaws of a suture passer similar to that shown in FIGS. 5A-5C. The tissue penetrator 65 and suture shuttle dock 66 are both visible on the first 62 and second 62′ jaws, respectively. FIGS. 6B-6G show side and perspective views of the lower jaw 62′. In this example, the lower jaw 62′ includes a textured tissue-contacting surface 68 that may help grasp tissue. In FIGS. 6B and 6F, an opening into the suture shuttle dock 66 is visible, as is a suture passage or guide, which is shown as a notch in the lower jaw through which the suture may extend when the suture shuttle is held within the suture shuttle dock.

As mentioned briefly above, the suture passer may also clamp or secure tissue between the jaws. Thus, the jaws may act as a clamp or gripper to secure the tissue. In some variations, the tissue is secured between the jaws before the suture is passed through the tissue. This may provide stability and enhanced accuracy in placing sutures. In some variations, the pressure to secure tissue between the jaws of the suture passer is regulated or limited so that the suture passer does not damage the tissue held between the jaws. For example, the suture passer may have a set point above which pressure applied by the jaws is limited. Thus, the suture passer may include a limiter to limit the pressure applied to the tissue between the jaws. The limiter may be a gear.

In some variations, the jaws may be activated or operated separately from the tissue penetrator. In general a tissue penetrator is configured to pierce the tissue held between the jaws to form a channel through the tissue through which the suture shuttle and suture may pass. In some variations, the channel through the tissue is formed within the tissue penetrator, and the suture shuttle passes through the tissue penetrator to be passed from one jaw to the other. Thus, the tissue penetrator may be a hollow structure (e.g., tubular) or it may include a compartment or cavity into which the suture shuttle may be held. In some variations the tissue penetrator is a solid (non-hollow) structure.

A tissue penetrator generally includes a sharp (tissue piercing) tip which may be moved through the tissue to form a passage therethrough. The tissue penetrator may mechanically form a passage through the tissue by pushing the sharpened end of the tissue penetrator through the tissue. In some variations, the tissue penetrator passes through the tissue after a channel or passage has already been formed through the tissue. For example, a passage through the tissue may be formed by the application of heat or energy (e.g., laser, ultrasound, etc.), or a separate mechanical element (needle, etc.). In some variations, the tissue penetrator includes a method of applying energy to form a passage through the tissue by non-mechanical means (e.g., laser, thermal, sonic, etc.).

In general, the tissue penetrator includes an attachment to releasable secure a suture shuttle. For example, the tissue penetrator may include a suture shuttle dock (which may also be referred to as a suture shuttle engagement region). The suture shuttle engagement region of the tissue penetrator may within the tissue penetrator (e.g., in a cavity or other opening formed within the tissue penetrator), or it may be on the outside of the tissue penetrator (e.g., around the perimeter of the tissue penetrator. The suture shuttle engagement region typically releasably secures the suture so that it can be held by the tissue penetrator until the tissue penetrator either engages the opposite jaw (e.g., a suture shuttle dock on the jaw opposite from the jaw to which the tissue penetrator is connected) or until it is triggered to be released (e.g., by a control such as a shuttle release control). The suture shuttle may be held by the suture shuttle engagement region mechanically (e.g., by friction), electromagnetically, by pressure (e.g., air pressure), etc. In some variations, the suture shuttle engagement region includes a lock which retains the suture shuttle in or on the suture shuttle engagement region.

In some variations, the tissue penetrator also includes a suture passage to at least partially guide the suture when the suture shuttle is engaged with the tissue penetrator. The suture passage (or suture guide) may help control the direction and orientation of the suture when the suture shuttle is held by the tissue penetrator (or suture shuttle dock).

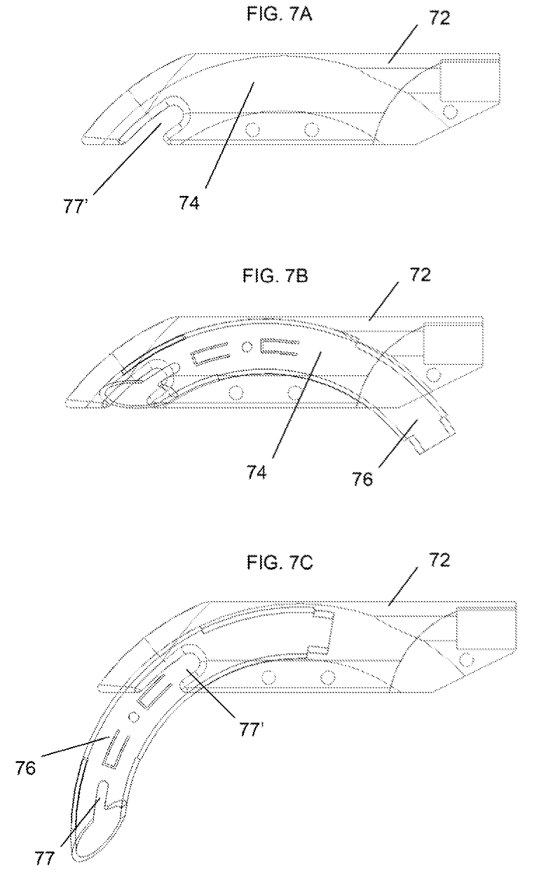

FIGS. 7B and 7C illustrate one variation of a tissue penetrator. FIG. 7A is one variation of a jaw 72 having a passage 74 into which a tissue penetrator 75 may fit, as shown in FIGS. 7B and 7C. In FIG. 7B the tissue penetrator 76 is shown retracted into the jaw 72. In this variation, the tissue penetrator is curved, so that it extends from the jaw at an angle. The distal end of the tissue penetrator in FIG. 7B is fully retracted into the jaw 72, causing the more proximal end of the tissue penetrator 76 to extend slightly from the jaw in the proximal direction. In some variations the tissue penetrator is completely surrounded by the jaw when it is retracted into the jaw. In some variations, the distal end of the tissue penetrator is sharp (e.g., pointed, serrated, etc.). Thus retracting the distal end of the tissue penetrator into the jaw may protect both the tissue and the tissue penetrator, and may enhance the ease of use of the device.

The tissue penetrator may be extended from the jaw so that it can extend distally and penetrate tissue held between the jaws. FIG. 7C shows a tissue penetrator 76 extended at least partially from the jaw 72. The tissue penetrator 76 and the jaw 72 both include a suture passage 77, 77′ to guide the suture when the suture shuttle is held within the device. This suture passage is shown as a notch 77, 77′ in both the tissue penetrator 76 and the jaw 72.

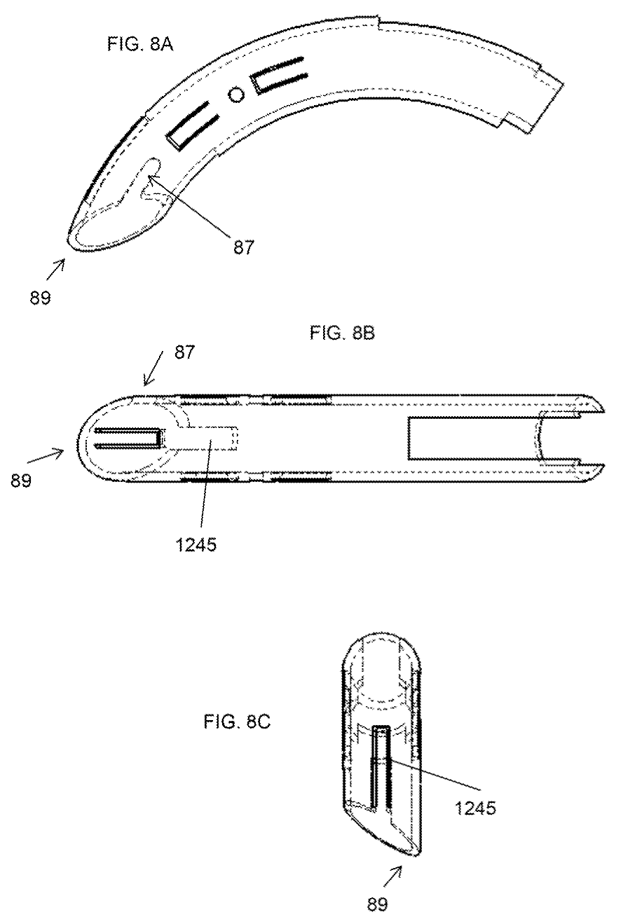

FIGS. 8A-8C show the tissue penetrator of FIGS. 7B and 7C in a side (FIG. 8A), top (FIG. 8B) and front (FIG. 8C) view. The distal end of the tissue penetrator 89 may include an edge to assist in penetrating the tissue. The suture passage 87 is visible. In some variations the edges of the suture passage are curved or blunted so that the suture is not damaged (or cut) when guided by the suture passage. In some variations, the suture passage is coated with a material (including friction-reducing materials) that help prevent damage to suture or help the suture to slide through the suture passage. In the tissue penetrator of FIGS. 8A-8C, the distal region end of the tissue penetrator may include one or more linkages for linking the tissue penetrator a tissue penetrator control. For example, the tissue penetrator may be connected to a wire or other pusher/puller for moving the tissue penetrator. In some variations the tissue penetrator may include gears or gear teeth (or voids to mate with gear teeth) so that the tissue penetrator can be extended and retracted.

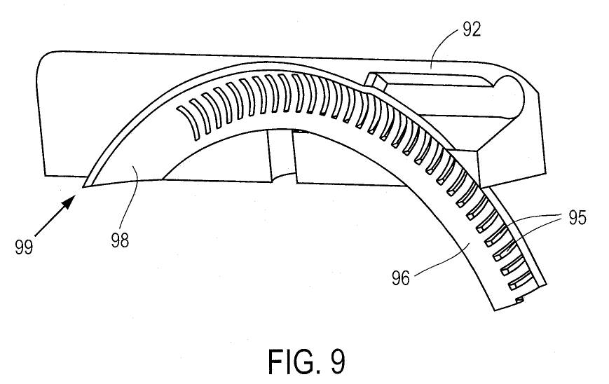

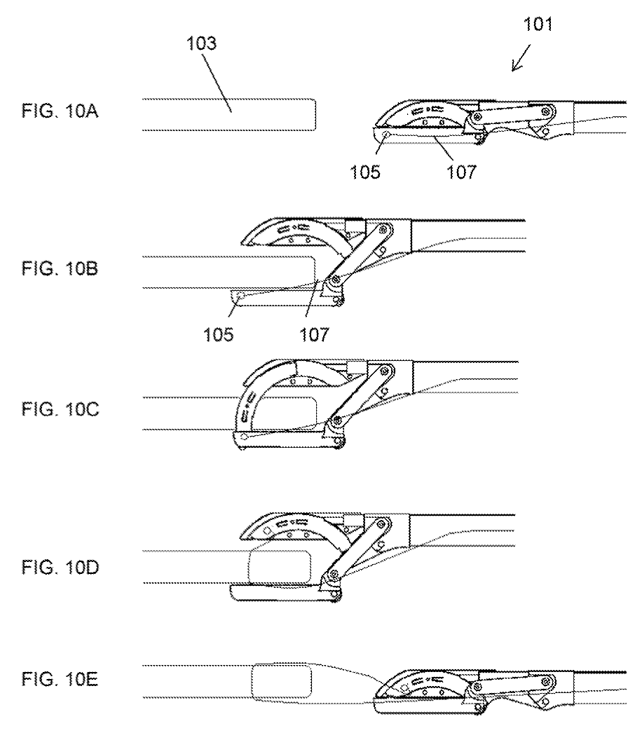

FIG. 9 is a three-dimensional view of a tissue penetrator 96 retracted within a jaw 92. This variation of a tissue penetrator is a curved needle or tube having a hollow region (at least at the distal tip) forming the suture shuttle engagement region 98. The distal end of the tissue penetrator 99 is configured to pierce tissue. For example, the distal end 99 may be sufficiently sharp so that it can penetrate biological tissue. The tissue penetrator shown in FIG. 9 is curved so that as it is extended from the jaw it will pass through any tissue between the jaws and contact the opposite jaw to transfer a suture shuttle. In some variations the tissue penetrator is bendable or flexible. For example, the tissue penetrator may be made of a shape-memory material (e.g., a nickel titanium alloy), or it may be hinged or otherwise shaped to bend. For example, the tissue penetrator may include cut out regions that allow it to bend or compress in a predetermined direction. In FIG. 9, the tissue penetrator 96 includes voids 95 for mating with a gear (e.g., a worm gear) along the tissue penetrator. Thus, the tissue penetrator may be extended or retracted (and/or locked in position) by interacting with these gearing voids 95. A gear (e.g., a spur gear, a helical gear, etc.) may be included within the jaw to move the device.

A tissue penetrator may be made of any appropriate material, particularly biocompatible materials. For example, the tissue penetrator may be made of metal, plastic, ceramic, or the like. In particular, the tissue penetrator may be made of a material that is sterile or sterilizable.

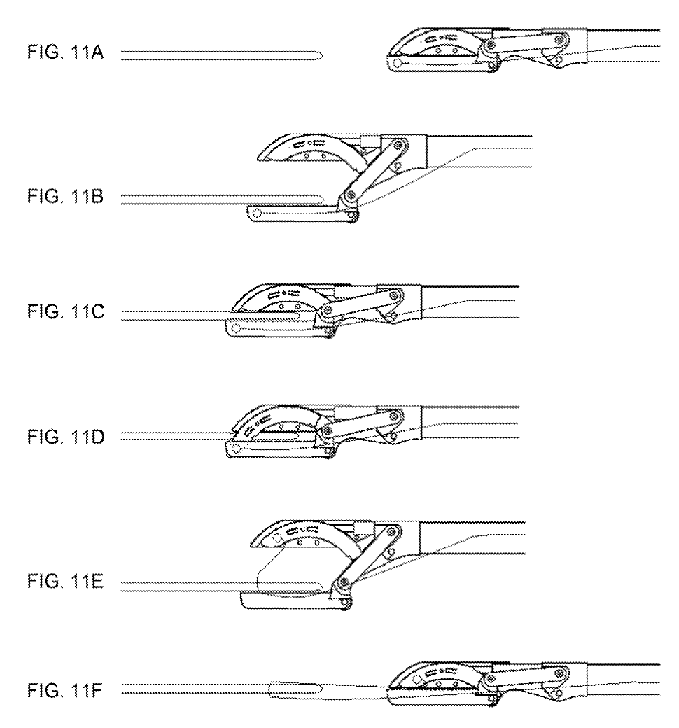

FIGS. 10A-10E and 11A-11E illustrate the operation of one example of a continuous suture passer. A suture passer 101 may be inserted into a subject's body using any appropriate technique. For example, a suture passer may be inserted into the body percutaneously using laproscopic or endoscopic techniques. When used as part of a laproscopic/endoscopic procedure, the suture passer may be dimensioned and configured for insertion through a catheter, introducer, or other small diameter structure. The diameter referred to is typically the diameter with the jaws in the “closed” position, taken perpendicularly through the long axis of the device. For example, the suture passer may have a diameter that is less than about 15 mm, less than about 10 mm, less than about 8 mm, or less than about 6 mm. In some variations, the diameter of the devices (with the jaws closed) is between 0.5 mm and 20 mm, or between about 3 mm and about 20 mm, or between about 5 mm and 20 mm. In addition, the diameter of the suture passer may vary along the length of the device. For example, the jaw region may have a slightly larger diameter than the intermediate region between the jaws and the handle. Generally the diameter refers to the maximum diameter of the portion of the device (in the closed configuration) that is inserted into a subject.

In FIG. 10A, the suture passer device is inserted and positioned with the jaws initially in the closed configuration. The suture passer in this example has been pre-loaded with a suture shuttle 105 connected to a suture 107, so that the suture shuttle is secured in the shuttle dock of the lower jaw. The suture may be guided proximally (so that it doesn't interfere with the suture passer, tissue, cannula or working portal) by a suture channel. Once the distal end of the suture passer 101 nears the tissue 103 to be sutured, the jaws may be opened wide enough that the tissue can fit between the jaws at the appropriate depth, as shown in FIG. 10B. In the variation of the suture passer shown in FIGS. 10A-10E and 11A-11F, the jaws open in parallel (as described above), which may allow the jaws to access regions of the tissue that are relatively distant from the edge of the tissue. Also, the jaws may be opened wider than suture passers whose jaws open scissor-like or otherwise form an angled opening. This is particularly useful for thick tissue, as shown in FIG. 10A-10E, but is also useful for thin tissues, as illustrated in FIGS. 11A-11F.

After the tissue is positioned between the jaws of the suture passer, the tissue can be pierced by the tissue penetrator, as shown in FIG. 10C. In some variations the tissue is first clamped between the jaws of the suture passer (as illustrated in FIG. 11C), which may stabilize the tissue between the jaws of the suture passer. The tissue penetrator may be moved to extend from the jaw or to retract back into the jaw. In some variations, the tissue penetrator may be retracted or extended by any means, including a worm gear, a push/pull wire or rod, a magnetic actuator (e.g., solenoid), a pneumatic actuator, etc. The tissue penetrator is extended from the upper jaw of the suture passer until it engages the lower jaw. In this example, the tissue penetrator extends at least partially into the lower jaw around the shuttle dock. Thus, the tissue penetrator may disengage the shuttle dock of the lower jaw so that the suture shuttle can be released from the shuttle dock of the lower jaw and into the shuttle dock (also referred to as the shuttle engagement region) of the tissue penetrator, as shown in FIG. 10C. The tissue penetrator can then be retracted back into the upper jaw, as shown in FIG. 10D, and the suture shuttle and attached suture is drawn through the tissue with tissue penetrator. After retracting the tissue penetrator with the suture shuttle, the suture passer can then be repositioned to pass another suture through the tissue by extending the tissue penetrator through the tissue, engaging the lower jaw, causing the shuttle engagement region to release the suture shuttle back into the lower jaw's shuttle dock. In the example shown in FIG. 10E, the suture passer can be withdrawn from the tissue after completing the single suture pass, and the suture can be tied, or again passed through the tissue.

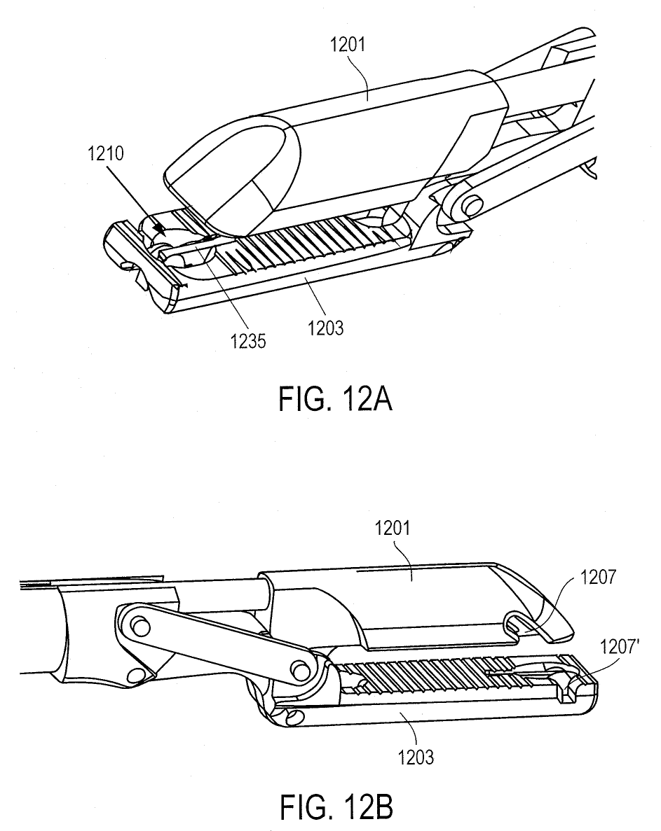

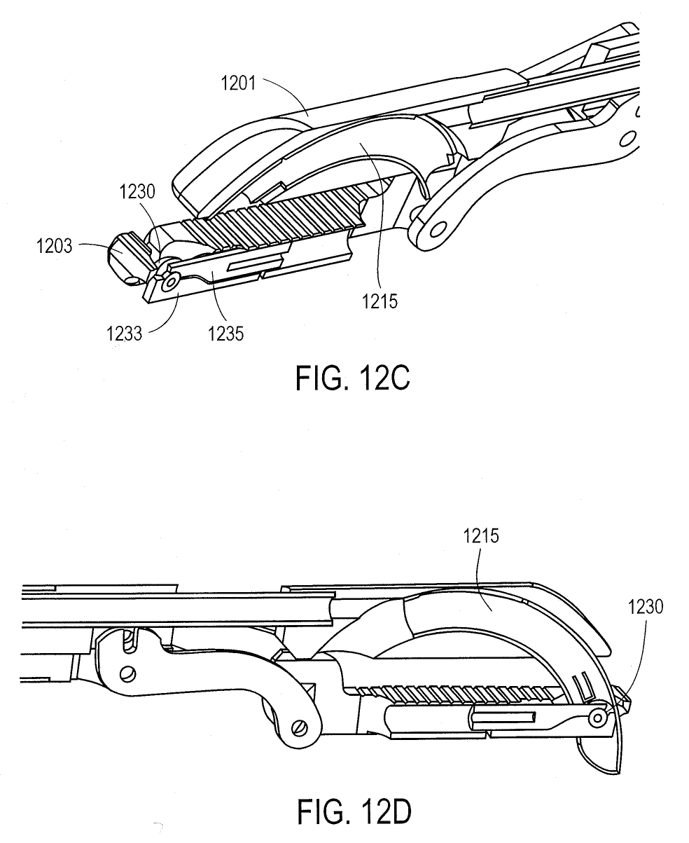

FIG. 11 shows the same suture passer being used to suture a substantially thinner tissue. The exemplary suture passer shown in these figures is configured so that as the jaws move, the relative positions of the tissue penetrator on the first jaw and the suture shuttle dock on the second jaw are substantially the same regardless of the position of the jaws. Thus, when the tissue penetrator is extended from the first jaw, it meets the shuttle dock on the second jaw. As described above, this may be accomplished by coordinating the compound motion of the jaws as they are opened and closed. FIGS. 12A-12D illustrate this coordinated motion in one variation of a suture passer.

FIGS. 12A and 12B are side perspective views of the jaws of a suture passer similar to the suture passer just described. A suture passage 1207, 1207′ is included in both the upper 1201 and lower 1203 jaws. The opening through the lower jaw into the shuttle dock 1210 is visible in the lower jaw 1203. Additional detail is apparent in FIGS. 12C and 12D, which show sections through the midline of the suture passers of FIGS. 12A and 12B. In FIG. 12C, a tissue penetrator 1215 is retracted into the upper jaw 1201. A suture shuttle 1230 (shown in cross section) is secured within the suture dock of the lower jaw 1203 by a shuttle retainer 1233 and a shuttle lock 1235. The shuttle retainer 1233 supports the shuttle 1230 in the shuttle dock, and the shuttle lock 1235 secured the shuttle in the shuttle dock. In this example, the shuttle lock 1235 and shuttle retainer 1233 engage a shuttle alignment feature that keeps the suture shuttle oriented within the suture passer. In this example the suture shuttle is a spherical suture shuttle having a circumferential channel that acts as the shuttle alignment feature. The shuttle lock 1235 and the shuttle retainer 1233 both fit within the circumferential shuttle alignment feature. Similarly, an engagement feature of the tissue penetrator 1215 (within the shuttle engagement region) fits within the shuttle alignment feature and may also help releasably secure the shuttle within the tissue penetrator, as shown in FIG. 12D.

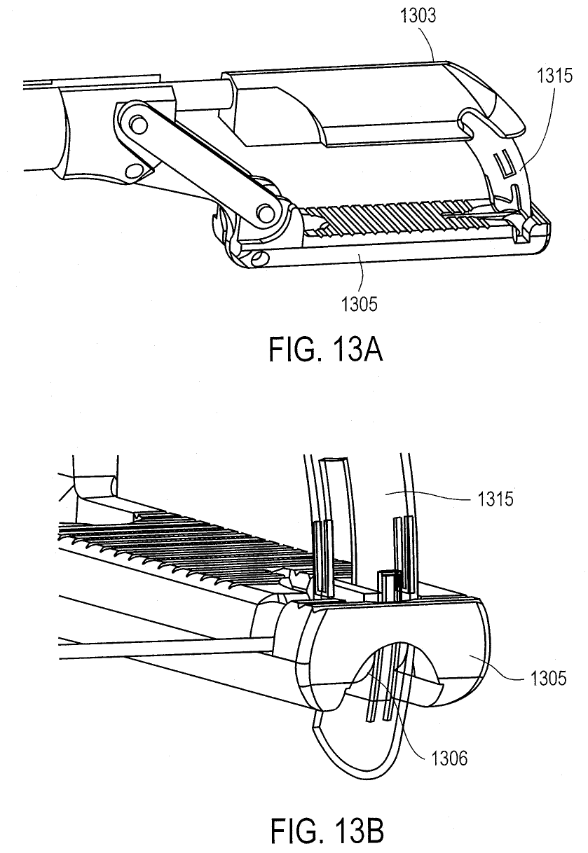

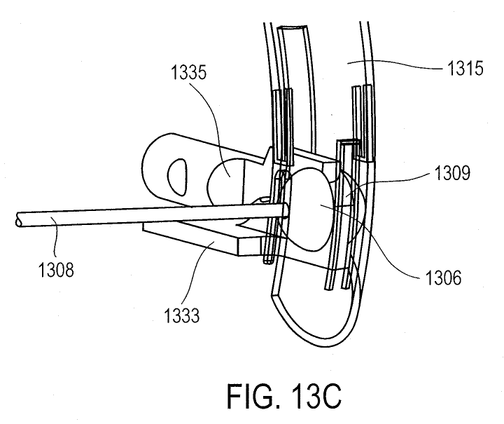

In FIG. 12D, the tissue penetrator is extended from the upper jaw, and passes at least partially through the lower jaw around the shuttle dock. Thus, the tissue penetrator 1215 in this example may include openings (or channels) for the shuttle lock 1235 and shuttle retainer 1233. These openings 1245 are apparent in the exemplary tissue penetrator shown in FIGS. 8B and 8C. FIGS. 13A-13C illustrate the interaction between the tissue penetrator, the suture shuttle, and the shuttle dock in the lower jaw. FIG. 13B shows the tissue penetrator 1315 and the shuttle dock in the lower jaw 1305 engaged with suture shuttle 1306. In FIG. 13C, the lower jaw 1305 has been removed, so that the shuttle retaining components of the shuttle dock in the lower jaw can be made visible. The shuttle retainer 1333 and the shuttle lock 1335 are securing a suture shuttle 13060 to which a suture 1308 has been attached. A tissue penetrator 1315 extending from the upper jaw 1303 has engaged the shuttle dock in the lower jaw where the tissue penetrator can engage the suture shuttle.

In the suture passer illustrated in FIGS. 12A-12D, the distal end of the tissue penetrator 1215 can be completely retracted into the upper jaw, as shown in FIG. 12A-12C. Thus, the device may be positioned in the tissue without having the tissue engage the tissue penetrator until the tissue penetrator is activated. This may prevent damage to the tissue, and make the device easier to manipulate with respect to the tissue.

The transfer of the suture shuttle between a shuttle dock in the lower jaw and a shuttle dock (or shuttle engagement region) in the tissue penetrator can be coordinated mechanically, electrically, magnetically, pneumatically, etc. For example, a reciprocating mechanical means may include one or more triggers that can toggle (open/closed) the shuttle lock in the shuttle dock of the lower jaw and/or a shuttle lock in the shuttle engagement region of the tissue penetrator. Thus, opening the shuttle lock in the lower jaw may close the shuttle lock in the upper jaw (tissue penetrator), and vice versa, allowing the shuttle to be transferred between the two. This toggling may occur when the two regions (the tissue penetrator and the shuttle dock of the lower jaw) contact or when the shuttle contacts both of these regions. In some versions only one shuttle lock is used (e.g. as part of the shuttle dock in the jaw). A shuttle lock may be any mechanism that prevents or helps to prevent the shuttle from leaving the shuttle dock (or shuttle engagement region). For example, a shuttle lock may be a mechanical lock (as shown in FIG. 12C) that mechanically engages or surrounds the shuttle, or it may be a magnetic lock. In some variations, the shuttle lock is a pressure lock (e.g., that applies suction to retain the shuttle).

Any appropriate suture shuttle may be used with any of the devices or systems described herein. In general, a suture shuttle include a suture attachment region that is configured to secure to a surgical suture, a shuttle body, and a shuttle retainer region that is configured to engage the suture passer. In some variations the suture shuttle (e.g., the body region) is substantially blunt. Thus, the suture shuttle does not itself penetrate the tissue, but relies on the tissue piercer of the suture passer to pierce the tissue. Thus, the outer surface of the suture shuttle may be smooth or soft.

The suture shuttle may be small (e.g., less than 5 mm in largest diameter). The size and shape of the suture shuttle may be related to the suture passer that it is to be used with, since the suture shuttle engages the suture passer (e.g., the tissue penetrator and the shuttle dock). In some variations, the suture passer is configured to be used with suture shuttles having different shapes and/or a range of different dimensions. For example, the shuttle dock and shuttle engagement regions of the suture passer may be oversized and include a size adapter. A size adapter may be, for example, a spring-biased holder that secures the suture shuttle within the shuttle dock regardless of the size of the shuttle. In some variations the shuttle lock is spring biased and acts as this holder.

A suture shuttle may also include an alignment feature for aligning the suture shuttle with respect to the suture passer. An alignment feature may be a surface feature (e.g., a groove, a notch, a knob, a protrusion, etc.), or an internal feature such as a hole or passage. In some variations, the shape of the suture shuttle acts as the alignment feature. For example, the suture shuttle may be irregularly shaped (so that it has an inherent orientation), or it may include one or more surfaces that mate with the suture passer in an oriented manner.

The suture attachment region of the suture shuttle is typically configured to secure a suture to the suture shuttle. In some variations, the suture attachment region is a passage or opening through the shuttle (or a region of the shuttle) through which the suture can be passed and secured. In some variations the suture attachment region is an outer surface of the suture shuttle to which the suture is attached. In some variations, the suture shuttle compresses the suture between one or more surfaces of the suture shuttle to secure the suture therein. For example, the suture attachment region may be a passage through the suture shuttle that can be crimped or otherwise clamped around the suture. In some variations the suture is secured to the suture attachment region by a glue or adhesive. The suture may also be attached to the shuttle by knotting, tying, etc. The suture attachment region may also be a loop or hook. In some variations, the suture may be preloaded to connect to the suture shuttle. In some variations, a suture that has already been implanted may be connected to a shuttle. For example, the suture may be connected by a hook or loop that can be threaded with the suture after the suture has been implanted into the subject's body. The suture may be attached (e.g., threaded) to a suture shuttle that is held by a suture passer as described herein, or it may be attached manually or by a separate suture threading device.

In general, a suture shuttle may be made of any material or combination of materials, particularly biocompatible materials. For example, the suture shuttle may be made of a metal, plastic, polymer, ceramic, etc. In some variations, the suture shuttle is made of a hard plastic. In some variations, the suture shuttle comprises a bioabsobable/biodegradable material. In some variations the suture shuttle comprises an elastic (e.g., rubber, silicone, etc.) material. Any appropriate suture may be used with the suture shuttle and suture passers described herein, including commercially available sutures (suture material and suture thickness may vary).

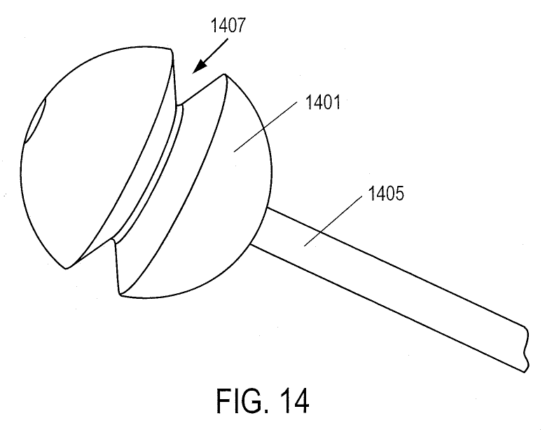

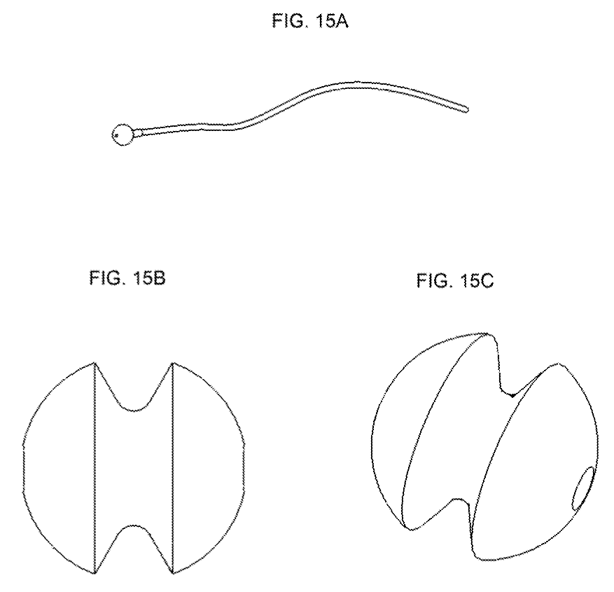

FIGS. 14-21B illustrate different variations of suture shuttles that may be used. For example, in FIG. 14, a generally spherical suture shuttle 1401 is shown. The suture shuttle includes a central passageway into which the suture is secured (e.g., the suture attachment region). As mentioned above, the suture 1405 can be threaded through the suture and the suture can be secured within the shuttle by crimping, adhesive or knotting. In FIG. 14, the suture shuttle also includes an alignment feature 1407 that is configured as a groove or channel around the outer perimeter of the shuttle. This alignment feature 1407 may also act as a shuttle retainer region, since it may engage a tissue penetrator so that the suture shuttle can be held (in an oriented fashion) by the suture passer, as illustrated in FIGS. 12C and 12D and FIG. 13C.

FIG. 15A shows another example of a spherical suture shuttle attached to a suture. FIGS. 15B and 15C are perspective views of a suture shuttle similar to the shuttle in FIG. 14, having a channel around the perimeter for aligning and interacting with a suture passer.

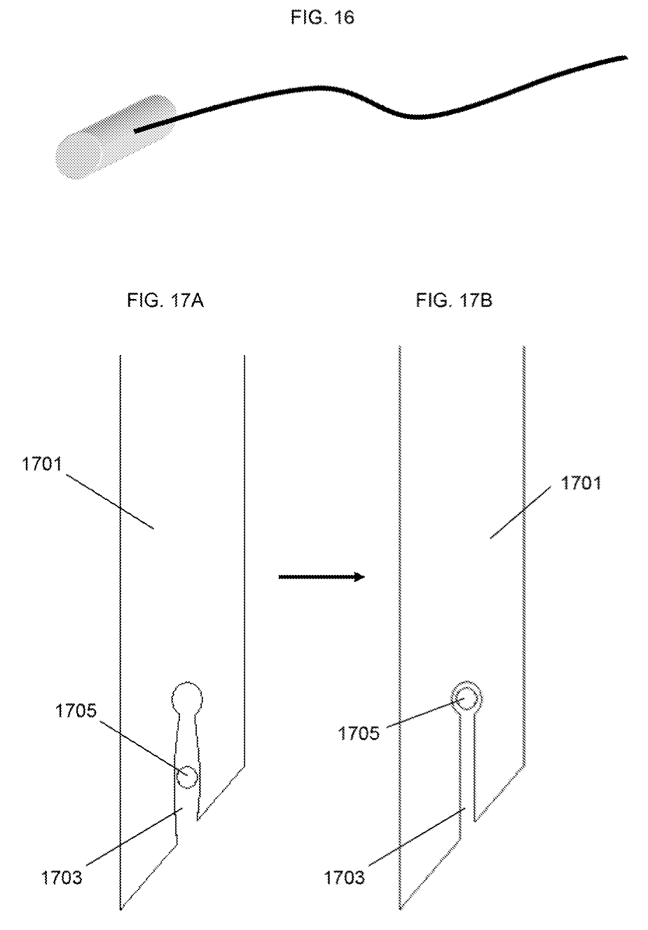

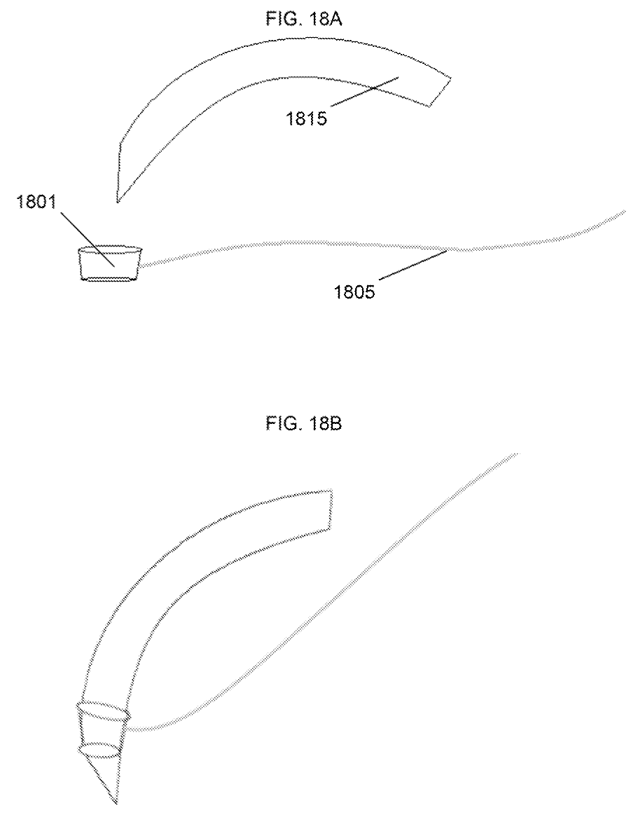

As mentioned above, the suture shuttle body may be any appropriate shape, including spherical, capsular, elongate, lobular, pyramidal, etc. In FIG. 16, a suture shuttle having a cylindrical shape is shown. The suture is connected to the cylindrical shuttle. In FIG. 16 the suture is attached perpendicular to the long axis of the cylinder, however, the suture shuttle may also be attached to the long axis of the cylinder. FIGS. 17A and 17B illustrate the interaction between a cylindrical shuttle such as the one shown in FIG. 16 and a tissue penetrator adapted for use with a cylindrical shuttle.

FIGS. 17A and 17B are cross-sections through a tissue penetrator 1701 and a cylindrical suture shuttle 1705. The tissue penetrator may be adapted for use with a cylindrical suture shuttle. As previously mentioned, the suture shuttle may engage the tissue penetrator by an interference (friction) fit. In FIG. 17A the tissue penetrator is a solid element having a slit 1703 cut therein. The slit provides a passage into which the cylindrical suture shuttle may pass. The width of this passage is slightly smaller than the thickness of the suture shuttle as the passage extends proximally up the tissue penetrator, however the most proximal region of the tissue penetrator may have a slightly larger diameter. Thus, the suture shuttle 1705 may be inserted into this passage 1703 as shown in FIG. 17A as the tissue penetrator is extended to engage a suture shuttle held within a shuttle dock. In some variations the walls of the channel into which the suture shuttle is pulled may deflect slightly to allow the suture to pass, as shown in FIG. 17A. Once the suture shuttle is completely seated in this shuttle engagement region of the tissue penetrator (FIG. 17B), the suture shuttle may be released by applying force to push the cylindrical shuttle back down the passage. For example, in a method similar to the embodiment shown above in FIG. 13A-13C, the tissue penetrator may engage a shuttle dock on the opposite jaw, and a shuttle lock that holds the suture shuttle in the shuttle dock may hold the shuttle therein while the tissue penetrator is retracted, leaving the suture shuttle in the shuttle dock.

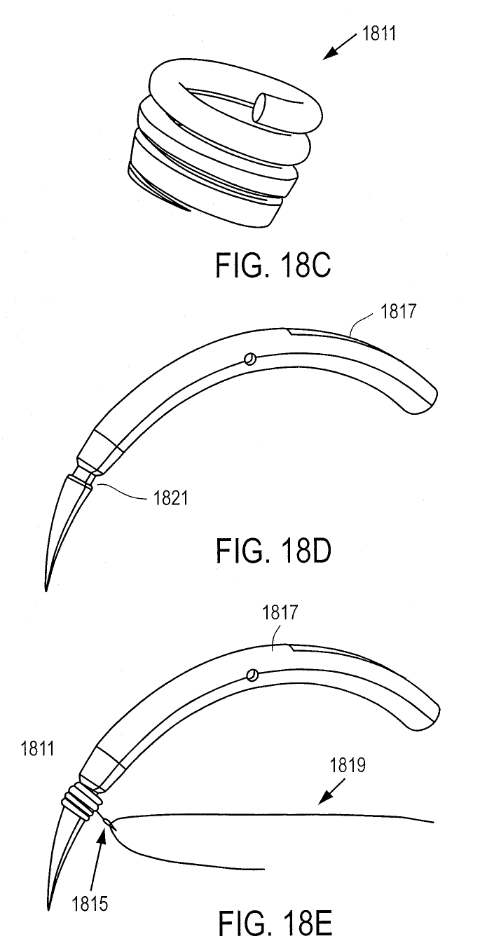

FIGS. 18A and 18B show another variation of a suture shuttle 1801 and tissue penetrator 1815. In this variation, the suture shuttle 1801 has an annular (e.g., cylindrical or tubular) body that may fit over the outside of the tissue penetrator 1815, as shown in FIG. 18B. A suture 1805 may be attached to the suture shuttle on the side of the shuttle, as shown. In some variations the suture shuttle is a spring or coil that may fit around the sides of the suture shuttle.

For example, FIG. 18C shows one variation of a suture shuttle 1811 having a shuttle body that is a ring or coil shape. This overall shape is substantially blunt, meaning that the shuttle body is not (by itself) sharp or tissue-penetrating. The suture shuttle shown in FIG. 18C also includes a suture attachment region (not visible in 18C, but apparent in FIG. 18E). The suture shuttle shown in FIG. 18C also includes a shuttle retainer region that engages a tissue penetrator, similar to the tissue penetrator 1815 shown in FIG. 18A. The shuttle retainer region is the inner surface of the ring or coil, since these variations of the suture shuttle typically engage with the outer surface of a tissue penetrator. As described above, the tissue penetrator may be a solid needle or a cannicular needle (e.g. having an opening therein), as illustrated in FIG. 18D. A ring or coil suture shuttle may be particular useful with small, solid (e.g., needle-like) tissue penetrators. For example, in FIG. 18D, the tissue penetrator 1817 may include a suture shuttle dock region (or shuttle engagement region) configured as a notch or ring 1821 along an outer surface of the tissue penetrator (e.g., slightly proximal to the distal tip of the tissue penetrator) that the suture shuttle may engage with.

In some variations, the suture shuttle body region is spring-like (e.g., in the coil embodiments). A spring embodiment may allow the suture shuttle body to more readily adapt to the size of the tissue penetrator, and to engage and disengage from the shuttle engagement region of a tissue penetrator.

In FIG. 18E, the suture shuttle is shown releasably attached to the tissue penetrator of FIG. 18D. The suture shuttle is connected to the shuttle body by an extension (e.g., a wire, string, etc.). The suture attachment region 1815 (described in more detail below) in this variation is a clip or loop through which a suture 1819 may be passed, having a progressively narrow opening for the suture.

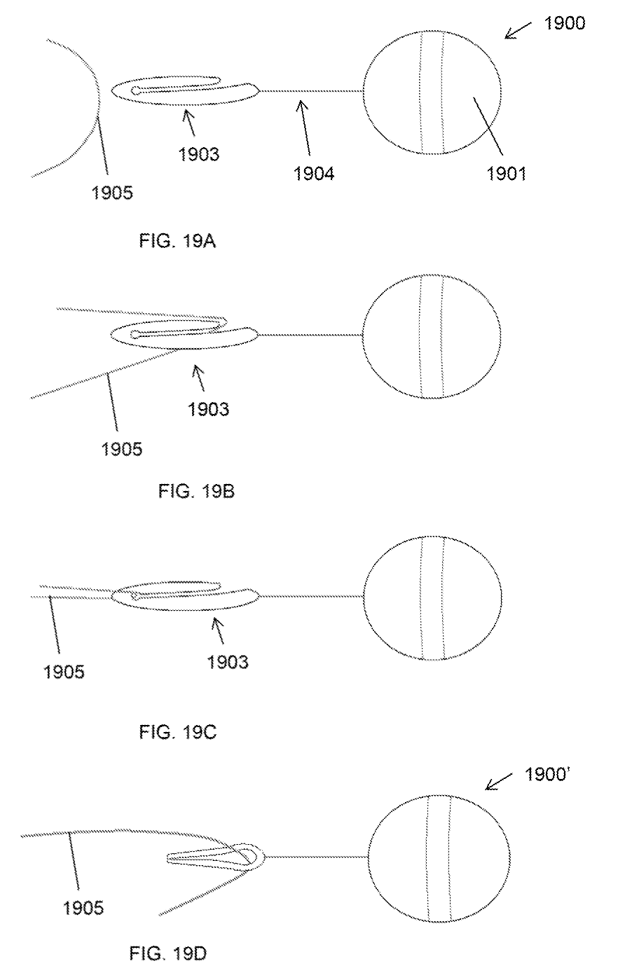

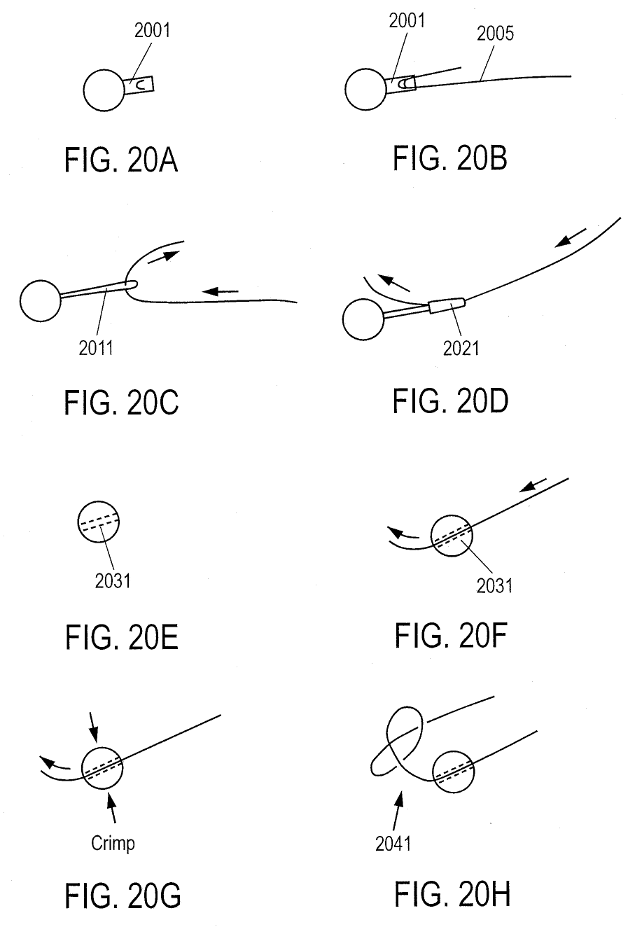

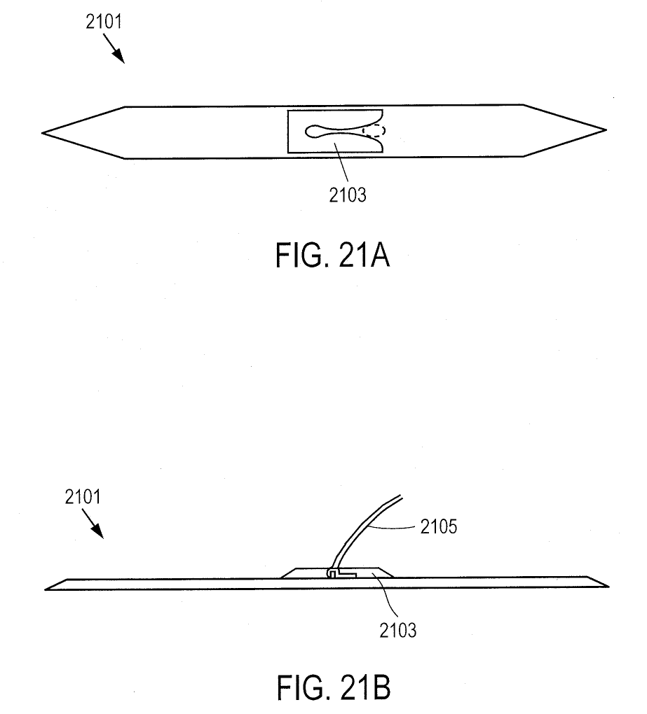

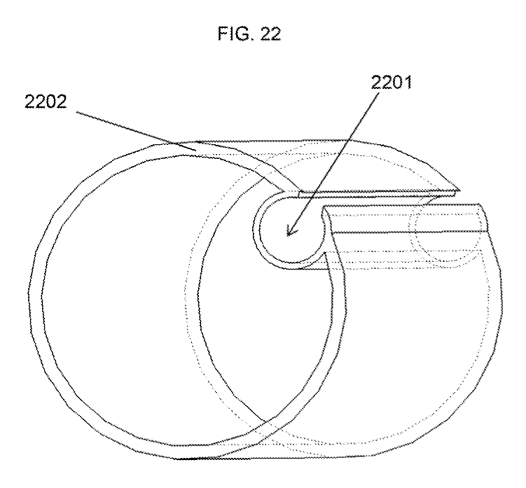

FIG. 19A-19C illustrates another method of attaching a suture to a suture shuttle. In FIG. 19A the suture shuttle 1900 includes cylindrical shuttle body 1901, as well as a suture attachment region 1903 that is connected to suture shuttle body 1901 by an extension 1904. The extension may be a wire, string, or the like, and may be part of the shuttle body, or it may be a separately attached region. The suture attachment region 1903 includes a channel into which the suture may pass. In some variations, the suture attachment regions secures the suture so that it does not readily slide once it is held by the suture attachment region. In some variations, the suture attachment region has a channel with a decreasing width; as the suture is pulled down the channel, the suture will become secured between the walls of the channel, and held in place. In some variations, the walls of the channel into which the suture is being pulled may deflect slightly to allow the suture to pass, as shown. In some variations, the end of the suture also includes a knot or lock that prevents it from being pulled out of the suture shuttle once it has been inserted.

A suture may be connected to a suture shuttle as indicated in FIGS. 19A-19C. The suture 1905 may be pulled through the channel in the suture attachment region 1903 until it is secured within the larger diameter region of the suture attachment region 1903. In this example, both ends of the suture 1905 extend from the suture shuttle. This may allow a suture to be passed while both ends of the suture passer remain behind. In some variations, one end of the suture passer may be knotted, or the suture may be tied, glued, crimped, etc. to the suture passer to prevent the suture from slipping out of the suture shuttle.

FIG. 19D shows another example of a suture attachment region 1903′ of a suture shuttle 1900′, configured as a pinched loop or clip. In this example, the suture shuttle attachment region 1903′ is a loop of material that is compressed closed at one end (e.g., the distal end). As the suture is drawn towards the pinched distal ends, the suture may become pinched by the loop, thereby securing the suture therein. In some variations, the suture shuttle attachment region includes ends that may be opened to release the suture if the force exceeds some threshold force. This may allow the suture to release from the suture shuttle before the force of pulling on the suture can damage the tissue.

FIG. 20A shows a suture shuttle having a protruding suture attachment region 2001. The suture attachment region includes a notch into which the suture may be drawn, as shown in FIG. 20B. Once the suture 2005 is connected to the suture attachment region (or the notch portion of the suture attachment region), the notch may be crimped or otherwise closed to prevent the suture from sliding out. FIG. 20C shows another variation of a suture attachment region 2011 configured as a loop. In this example, the suture may be threaded through the loop 2011, as indicated by the arrows. Once the suture has been threaded through the suture attachment region, it may be secured in place by knotting, etc. FIG. 20D illustrates a suture shuttle having a tubular suture shuttle region 2021, into which a suture shuttle may be threaded, as indicated by the arrows.

FIGS. 20E and 20F show another variation of a suture shuttle in which the suture attachment region is a channel 2031 through the body of the suture shuttle. As previously mentioned, the suture may be threaded through the channel, and then secured within the suture shuttle. FIGS. 20G and 20H illustrate two different methods of securing a suture within the suture shuttle. For example, in FIG. 20G the suture is secured within the channel by compressing the outer perimeter of the suture shuttle to crimp it, compressing the suture within the channel and preventing it from exiting. FIG. 20H illustrates another variation, in which the suture is passed through the device and then knotted 2040 after passing through the suture attachment region.

FIGS. 21A and 21B illustrate another variation of a suture shuttle. In this example, the suture shuttle is not blunt, but is pointed at either end, so that the suture shuttle may be the tissue penetrator (or may be part of the tissue penetrator) for a suture passer. A variation of a suture passer implementing a sharp suture shuttle is described in greater detail below. In the top view of the suture shuttle 2101 shown in FIG. 21A, the suture attachment region 2103 is located in the center of the suture shuttle. The suture attachment region includes a slot having a narrow channel and wider opening region, as described previously in FIGS. 17A and 17B. A suture 2105 may be attached to the shuttle by first knotting the suture (or otherwise forming a larger region of the suture), and sliding the suture into the channel until it reaches the larger diameter opening. The suture 2105 can then be pulled until the end of the suture (knot or larger region) is secured. This is illustrated in the side view of FIG. 21B.

As previously mentioned, the suture shuttle may include an orientation feature for orienting the suture. In addition (or alternatively), the suture passer may include one or more suture passage or suture guide for directing the suture extending from the suture shuttle. In general, a suture passage may include a channel into which the suture may reside, to protect the suture from interfering with the device. A suture channel may guide the suture as is exits the distal end of the device (as described above). A suture channel may also be included in the intermediate region of the device (e.g., the region between the distal end and the proximal end. For example, a suture channel may be included as a channel along the side of the intermediate region of the device. FIG. 22 shows one variation of a suture channel 2201 that may be included as part of the intermediate region 2202 of the device. In FIG. 22, the suture channel is an open channel that may extend at least partially along the length of the device. The channel is open along the distal-proximal axis of the device, so that the suture may be inserted into the channel during operation of the device. A suture channel can both guide the suture and protect the suture, allowing it to move smoothly during operation of the device. A suture channel may be particularly useful for suture passers that are used as part of a laproscopic or endoscopic procedure, in which the suture passer is inserted into a cannula or introducer that might otherwise inhibit the free motion of the suture.

Any of the suture passers described herein may also include a handle for manipulating the distal end (e.g., jaws) of the suture passer. This handle may include one or more controls such as jaw controls (for moving one or both jaws), tissue penetrator controls (for controlling the extension and/or retraction of the issue penetrator), jaw locks (for locking the jaws in a position), tissue penetrator locks, etc., tip deflection controls (e.g., for deflecting the distal end of the device), or the like. These controls may be levers, dials, buttons, sliders, switches, screws, knobs, or the like.

FIGS. 23A and 23B are schematic illustrations of different handles that may be used. In FIG. 23A, the handle is configured as a hand grip having a jaw control and a tip deflection control. In this example, the jaws may be opened or closed by applying pressure to squeeze the handle 2301 in the large grip region 2303. I some variations, the jaw control 2301 is a proportional control. Thus, the jaw (or both jaws) may be moved in proportion to the squeezing of the handle. The control may be biased (e.g., by a spring 2307) to return the jaws to the closed (or open) configuration, or to increase the force necessary to open or close the jaws. In some variations the same control may be used for both the jaw control and the tissue penetrator control. For example, in FIG. 23A, force may be applied to the handle control to close the jaws. As the jaws close, the force of the closing may be fed back into the control, so that above a threshold for closing, the jaws are no longer closed, but instead the tissue penetrator is deployed.

In FIG. 23A, a separate tissue penetrator controller 2311 is used, rather than a combined jaw control 2301′ and tissue penetrator control 2311. For example, in FIG. 23A, the index and middle finger may be used to control the tissue penetrator control 2311. In some variations the tissue penetrator is deployed by triggering, which automatically extends the tissue penetrator, exchanges that suture shuttle, and retracts the tissue penetrator. In some variations, the tissue penetrator is be deployed in a proportional manner, similar to the opening/closing of the jaws described above.

The handle may also include a control for ejecting the suture shuttle from the device, or for loading the suture shuttle into the device. For example, a control may be used to disengage the shuttle lock of a shuttle dock or shuttle engagement region (e.g., in a tissue penetrator). The handle may also include controls for reloading a suture shuttle into the distal end of the device. In some variations, the device may be configured so that a cartridge of suture shuttles (either with or without sutures attached) may be used. For example, a plurality of suture shuttles may be included as part of a cartridge that is loaded into one of the jaws. As a suture shuttle is passed, if may be ejected, and a new suture shuttle may be presented into the shuttle dock to take its place.

In some variations the handle is configured so that the device may be operated by a single hand. For example, the jaws may be positioned and the suture may be passed between the jaws by using only one hand. In some variations, the handle may be configured so that both hands may be used.

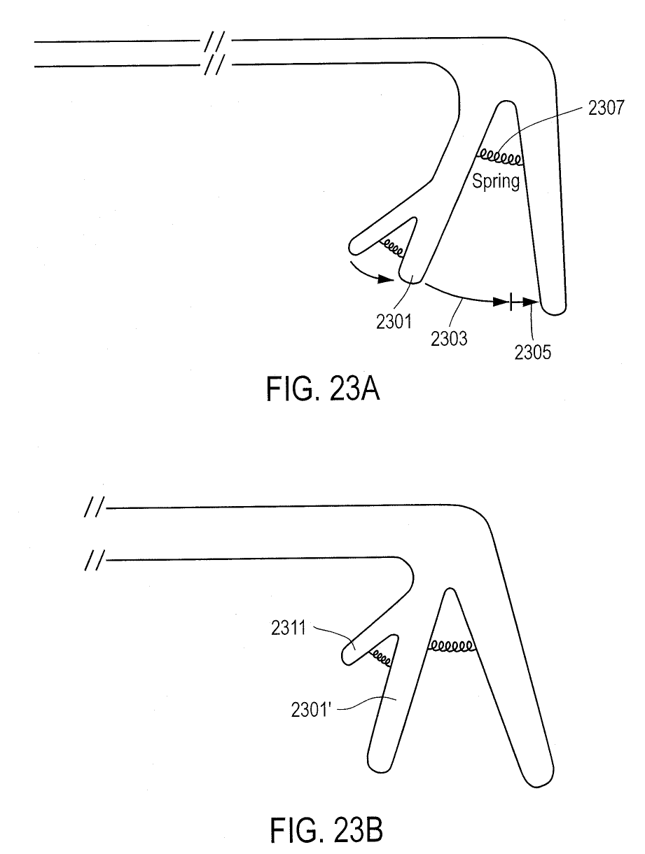

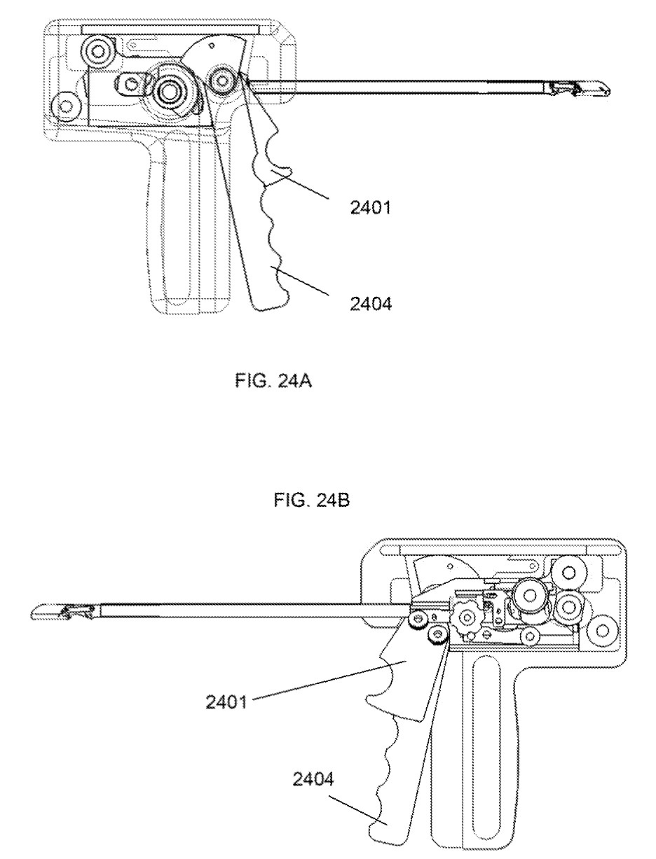

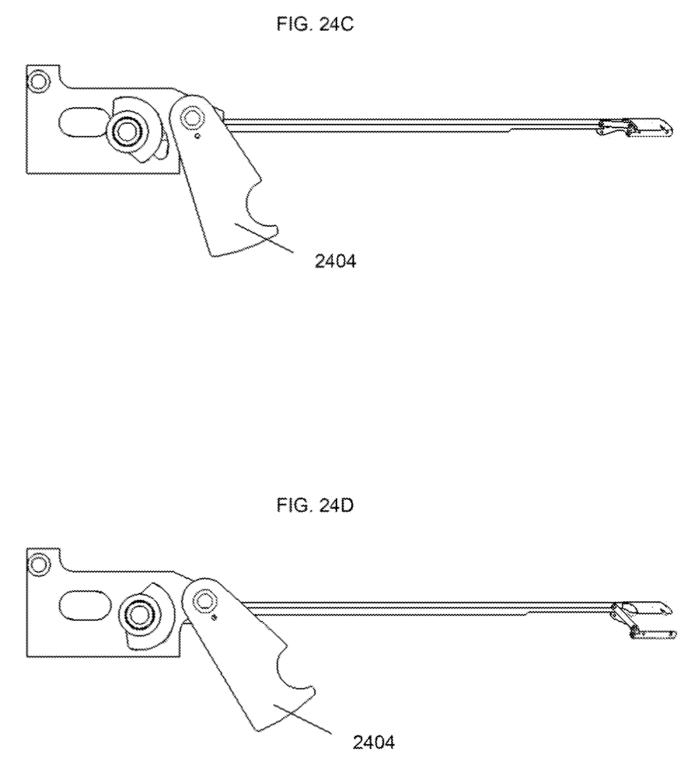

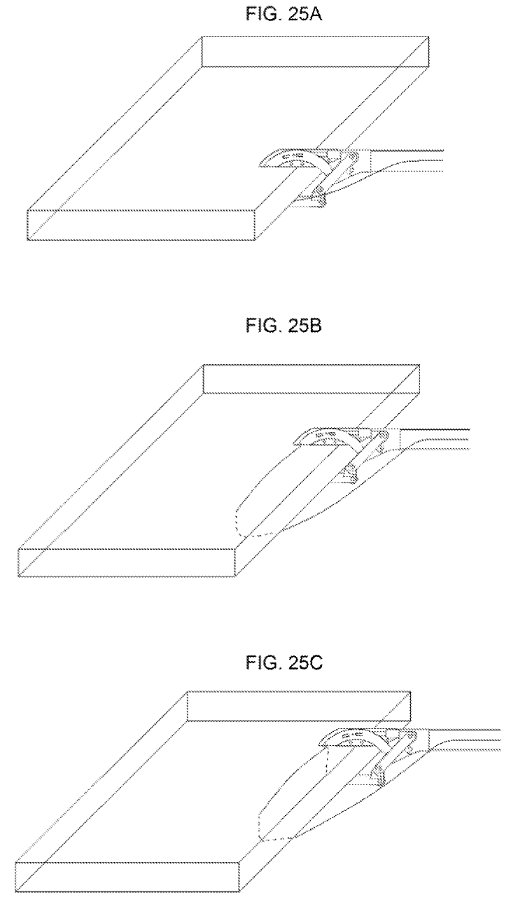

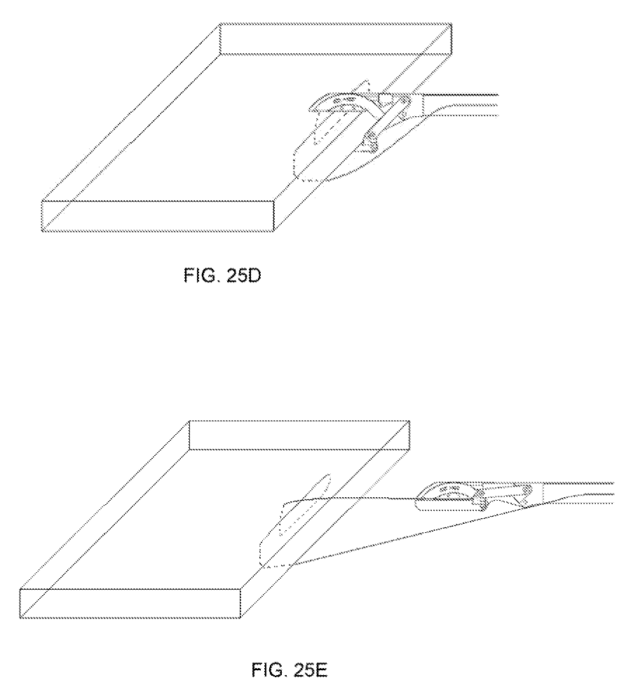

The handle may include a handle housing. The housing may cover the inner workings of the handle and controls. FIGS. 24A and 24B illustrate one variation of the device in which the controls and the mechanism for operating the controls are visible. For example, the jaw control may regulate the reciprocal movement of the jaws by connecting the handle control 2401 to the first (upper) and second (lower) jaws using gears and a cable or pusher (not visible). Thus, as the jaw control (handle) is moved, this movement is converted into movement of the upper and lower jaws to open and close. FIGS. 25A and 25B also illustrate this. FIGS. 25A and 258 show some of the components illustrated in FIGS. 24A and 24B, particularly those components used to open and close the jaws as previously described.

The mechanisms illustrated in FIGS. 24A and 24B also include the mechanisms used to extend and retract the tissue penetrator. In the exemplary device shown in FIG. 24, the tissue penetrator may be retracted completely into the upper jaw. A separate tissue penetrator control 2404 may be used to extend or retract the tissue penetrator. In some variations, the gearing for extending and retracting the tissue penetrator is configured (within the handle) to be moved with the jaw control gearing, so that the motion of the tissue penetrator may be coordinated with the motion of the jaws as they open and close. In some variations the motion of the tissue penetrator is mediated at least in part by a pusher or cable. In some variations the motion of the tissue penetrator is mediated by a worm screw.

The handle housing may also include a control mechanism for regulating the passage of the suture shuttle between the jaws. In some variations the suture shuttle is releasably secured in either the first or second jaw (e.g., the tissue penetrator or the shuttle dock) by a shuttle lock holding the suture shuttle within the shuttle dock. By coordinating the shuttle lock so that it engages the shuttle in the shuttle dock on every other complete motion of the tissue penetrator, the shuttle can be passed between the shuttle dock and the tissue penetrator. Thus, the lock (or locks) securing the suture shuttle may be controlled to alternatively engage or disengage. The suture passer may also include a manual over ride (e.g., on the handle) to eject the suture shuttle from the device.

The handle may also house additional mechanisms, including tip deflecting mechanisms (e.g., one or more cables or pushers for deflecting the distal end of the device), lighting sources/cameras (which may be used to help visualize the distal end of the device), fluid channels or adding/removing fluid, or the like.

In some variations, the tissue penetrator includes logic or controllers for regulating the activation of various aspects of the device, including the jaws, the suture shuttle location, and the tissue penetrator. A controller may be an electronic controller, and may include software or hardware (or both). For example, the tissue penetrator may include a controller for regulating the force applied by the jaws on the tissue. A controller may also regulate the motion of the tissue penetrator. For example, the maximum threshold of force applied to the tissue may be regulated by a controller. A controller (e.g., a micro-controller chip) may be used. In some variations one or more force sensors may be included. The device may sense the force applied by the jaws on the tissue, or by the tissue penetrator on the tissue (or both) and may adjust the maximum force applied to the tissue by the jaws. Sensors may also be used to determine the force applied by the tissue penetrator. Since the force required to penetrate the tissue may be variable, the device may include a controller that receives inputs from one or more sensors, and sets the triggering and/or the force applied by the tissue penetrator accordingly.

As briefly mentioned above, the devices described herein may be used to suture a tissue by making a suture stitch, and by tying or knotting a stitch. FIG. 25A-25E illustrates one variation of a method for making a stitch in tissue using the devices described herein. This example illustrates the forming of a modified Mason-Allen stitch. In FIG. 25A, the suture passer device is first positioned near the tissue, and the jaws are opened so that the tissue may be fit between them. The jaws of the suture passer may be closed (e.g. clamped) onto the tissue, and the tissue penetrator (not shown) may be passed through the tissue. In one variation, the suture passer is first loaded with a suture shuttle and suture in the lower jaw. When the tissue penetrator passes through the tissue and engages with the shuttle dock in the lower jaw, the interaction between the tissue penetrator and the shuttle dock triggers the lock holding the suture shuttle within the shuttle dock to open, and the shuttle may be engaged (via a friction fit) to the with the suture passer's shuttle engagement region, and withdrawn through the passage formed by the tissue penetrator, as shown in FIG. 25B. The suture passer may then be repositioned, as shown, and the suture and suture shuttle again passed back to the lower jaw, as seen in FIG. 25C. In this example, the suture shuttle and suture are passed, via the tissue penetrator, through the tissue and into the second (lower) jaw and in close proximity to the shuttle dock. The interaction between the tissue penetrator and the suture clock again toggles the shuttle lock so that it engages (at least indirectly) with the suture shuttle, holding it in the shuttle dock so that the tissue penetrator can be withdrawn, leaving the suture shuttle in the lower jaw once again. These steps may be repeated as often as necessary to continuously stitch through tissue and other materials, as partially illustrate in FIGS. 25D and 25E.

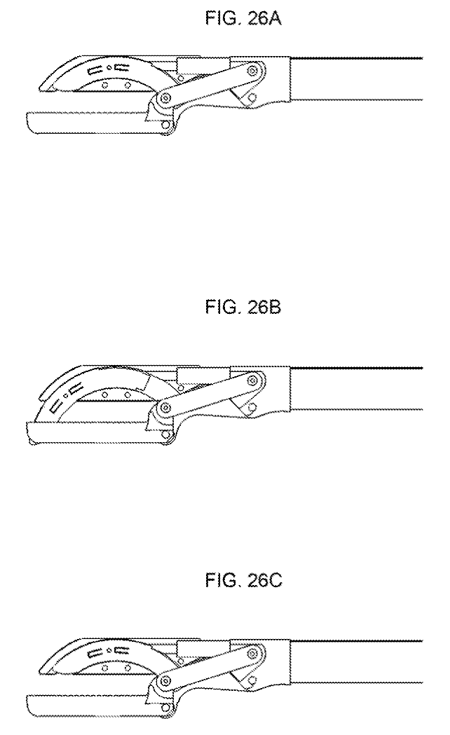

FIG. 25E, which shows the last step in making a modified Mason-Allen stitch, also suggests one way in which the suture passer may be used to form a knot by passing the suture over another region of the suture and drawing the suture taut. This process is also described in FIG. 26A-26C, in an example in which the suture is passed even when tissue is not present between the jaws. In FIG. 26A, the jaws are first be locked into position so that there is a separation between the jaws. For example, a jaw lock may be used to hold the jaws in the partially-opened configuration. If the user would like to form a knot, the suture passer may be positioned so that the suture shuttle and attached suture will be passed behind the rest of the length of the suture. The tissue penetrator may then be extended (even when there is no tissue between the jaws, as shown in FIG. 26B), and the suture shuttle and suture may be passed between the jaws. Once the suture is passed between the jaws, the suture behind the suture passer will have formed a loop and the end of the suture will have passed through the loop as the suture is passed between the jaws. This loop may then be tightened to knot the suture, and/or the suture passer may be withdrawn or additional sutures may be made.

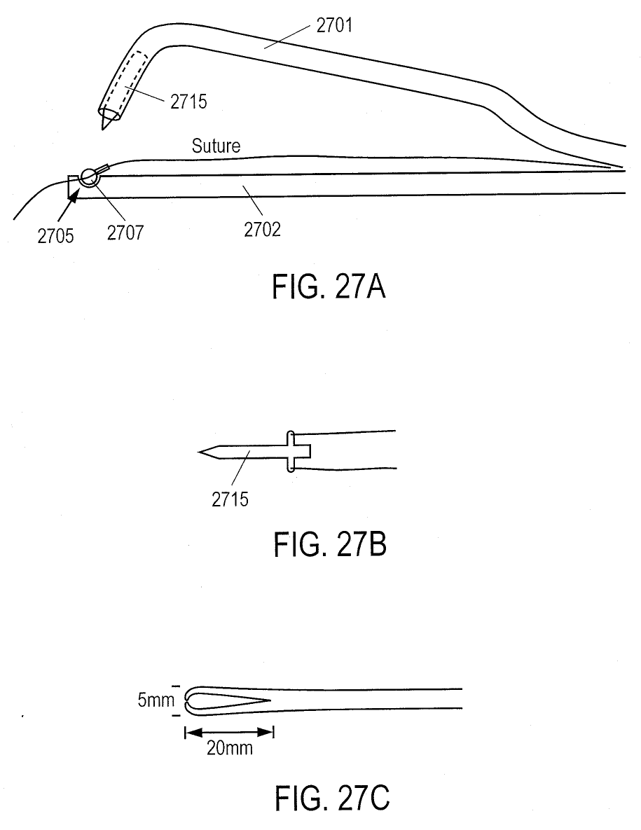

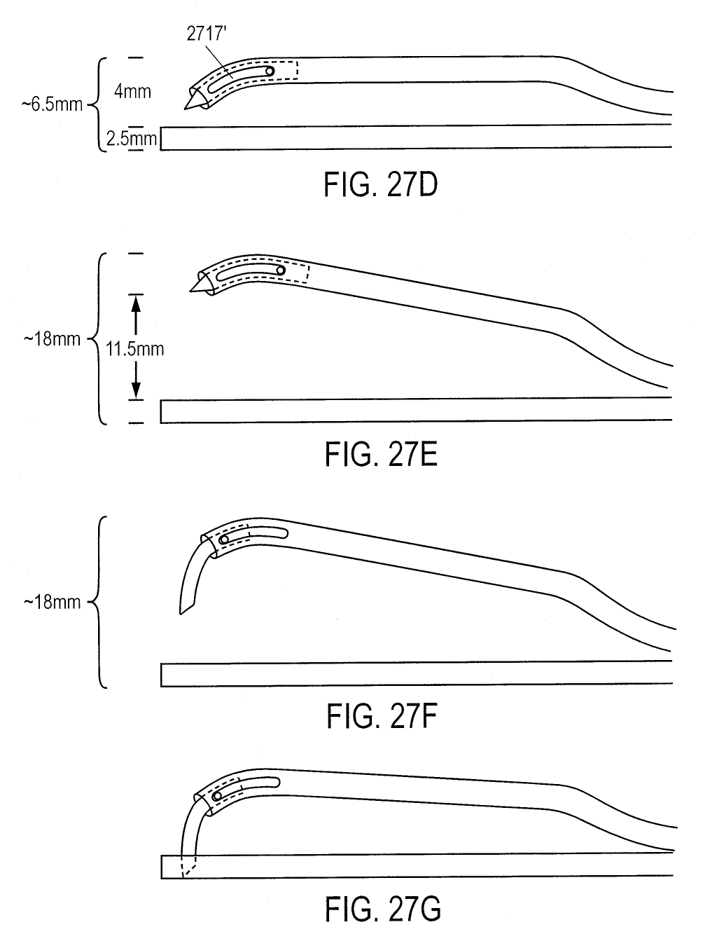

FIGS. 27A-35 illustrate different variations of the suture passer devices described herein. As mentioned, any of the elements or features described in these examples may be used with any of the other embodiments described herein.

FIG. 27A shows the distal end of a suture passer having a first jaw 2701 and a second jaw 2703. A tissue penetrator 2715 is retracted in the first jaw, and the second jaw 2703 includes a suture shuttle dock 2705 in which a suture shuttle 2705 rests. In this variation of a suture passer, the upper and lower jaws open and close by moving scissorlike around a single pivot point. Thus, the first and second jaws do not open substantially parallel, as previously described. The tissue penetrator is configured so that it can extend from the upper jaw and abut (or engage) the shuttle dock in the lower jaw, so that the suture shuttle may be passed between the two. The tissue penetrator in this example is not substantially curved, but is straight. FIG. 27B shows a perspective view of a tissue penetrator 2715.

In some variations of a tissue penetrator, the proximal end of the tissue penetrator includes one or more attachment sites for manipulators that can extend or retract the tissue penetrator. For example, in FIG. 27B the tissue penetrator 2715 is connected to two cables that extend proximally and may be coupled to a tissue penetrator control to extend or retract the tissue penetrator. In some variations, the tissue penetrator also includes one or more guides for guiding the motion of the tissue penetrator as it extends or retracts. In FIG. 27B the tissue penetrator includes a guide configured as two pegs that stick out from the sides of the distal end of the tissue penetrator and can engage with a track or rail in the first jaw to guide (or limit) the motion of the tissue penetrator.

A tissue penetrator may be any appropriate length. In general, the tissue penetrator may be is slightly longer than the opening width of the jaws (in variations have two jaws). Thus, the tissue penetrator may be long enough to penetrate tissue even when the jaws are open. In some variations, the jaws may be opened to approximately twice the diameter of the closed jaw. For example, the jaws may be opened so that the space between the jaws is greater than 10 mm, greater than 15 mm, greater than 20 mm, greater than 25 mm, greater than 30 mm, or greater than 40 mm.

FIG. 27C illustrates the scale of one variation of a suture passer similar to the example shown in FIG. 27A. In this example, the suture passer has two jaws that project distally from the end of the device. When the jaws are closed (as shown), they have a maximum diameter of approximately 5 mm. The jaws are approximately 20 mm long. In general, a suture passer may have a larger or smaller diameter, and the length of the jaw (or jaws) may be smaller or longer than 20 mm. For example, the distal end of the device may have a maximum diameter of between about 20 mm and about 3 mm. In some variations, the diameter of the distal end of the device is less than about 15 mm when the device is closed. In some variations, the diameter of the distal end of the device is less than 12 mm, less than 10 mm, less than 8 mm, or less than 6 mm. Similarly, the jaws may be any appropriate length. For example, the first and second jaws may be less than about 30 mm long, less than 27 mm long, less than 25 mm, less than 20 mm long, etc. The elongate neck region may have a diameter that is less than the diameter of the closed first and second jaws.

FIGS. 27D-27G illustrate the operation of a suture passer that is similar to the suture passer shown in FIG. 27A, in that the jaws open and close from a single pivot point (e.g., the jaws do not remain substantially parallel to each other as they open and close). In FIG. 27D the suture passer is shown in substantially closed position, with the tissue penetrator 2715′ retracted. As described before, the tissue penetrator includes one or more guides for guiding the motion of the tissue penetrator as it leaves the upper jaw. In this example, the tissue penetrator includes a post that is movable in a channel in the side of the upper jaw.

In FIG. 27E the jaws have been opened, so that the space between the jaws is greater than about 11 mm. In this example, the jaws are opened by retracting the upper jaw from the lower jaw. In FIG. 27F the tissue penetrator 2715′ is extended from upper jaw towards the lower jaw. In some variations, this tissue penetrator is curved or bendable, so that the sharp tip of the tissue penetrator may extend towards the lower jaw (and particularly the shuttle dock in the lower jaw where a suture shuttle may be seated). For example, the tissue penetrator may be jointed to bend. In some variations the tissue penetrator is made at least partially of a shape memory material so that it can assume a bend shape upon leaving the upper jaw. In some variations, the tissue penetrator is rotated as it is extended, so that the tip of the tissue penetrator is moved towards the lower jaw.

In the example shown in FIGS. 27D-27G, the tissue penetrator is shorter than the 11 mm length that the jaws are opened. Thus, the upper jaw may be closed to bring the tissue penetrator in proximity to the shuttle dock in the lower jaw, as shown in FIG. 27G. The interaction of the tissue penetrator and the shuttle dock (as well as the suture shuttle) may pass the suture shuttle between the jaws, as previously described.

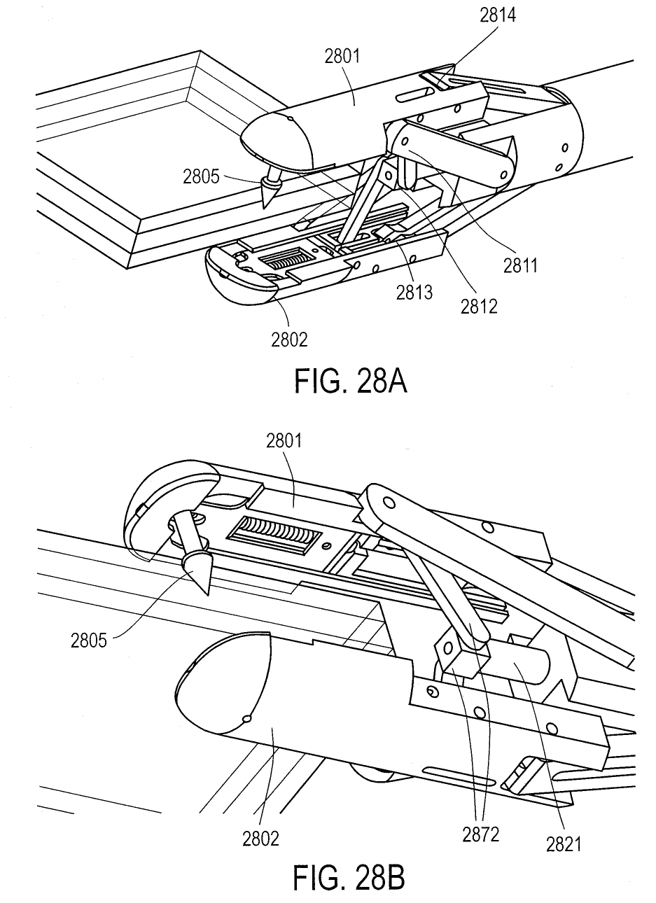

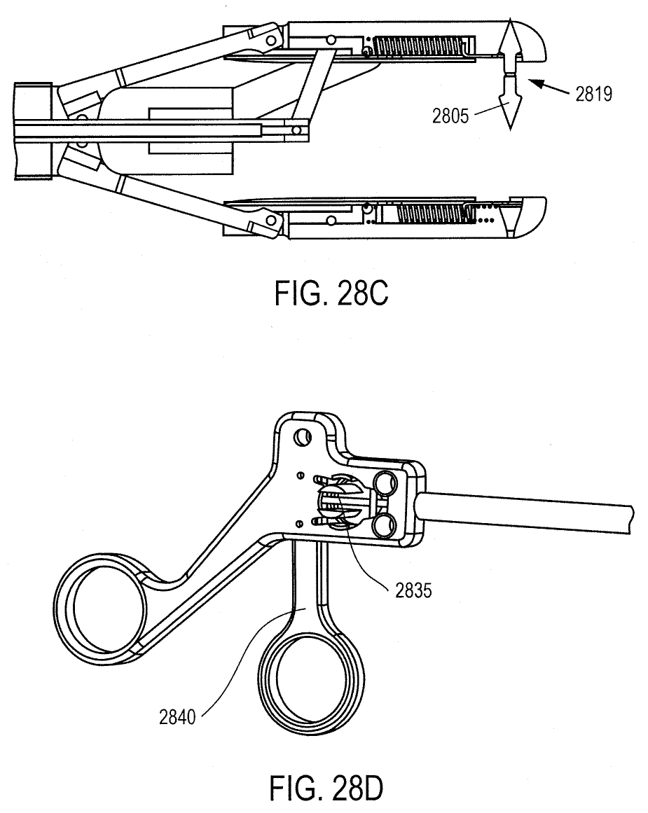

FIGS. 28A to 28D show another variation of a suture passer and a suture shuttle in which the suture shuttle is the tissue penetrator, and a separate tissue penetrator is not connected to one or the other (or both) jaws. FIGS. 28A and 28B show side perspective views of this variation. The suture passer device includes two jaws 2801, 2802 that are configured to open and close so that the jaws are substantially parallel, as previously described. In this example, each jaw includes multiple pivot joints 2811, 2812, 2813 that allow the jaw to open and close in a parallel manner. For example, the upper jaw may be hinged at the proximal end of the jaw 2814 and also near the middle of the jaw 2811. As the jaws are opened, the dual constraints of the proximal and middle hinges result in the upper jaw opening in parallel to the similarly hinged lower jaw. In this example, the jaws may be opened and closed by driving a pusher 2821 that is pivotally hinged to the upper and lower jaws 2812, as indicated in FIG. 28B.

The suture shuttle 2805 in FIG. 28A-28D is a tissue-penetrating suture shuttle, similar to that shown in FIGS. 21A and 21B. The tissue-penetrating suture shuttle is sharp on both ends, and includes a central suture attachment region 2819 that is visible in FIG. 27C. In this example, both the upper and lower jaws include a suture shuttle dock that is configured to secure the suture shuttle. In this example, the suture shuttle may be passed by compressing the jaws of the suture passer to alternately engage and disengage the sharp suture passer between each jaw. Although the suture passer protrudes from the jaws in this variation, which may interfere with ability to grasp the tissue, the parallel-opening of the jaws may permit the jaws to be opened widely.

FIG. 28D shows one variation of a handle for the device, including a jaw control lever 2840. The handle in FIG. 28D also shows a portion of a shuttle lock alternator 2835 that can control the alternating gasping/releasing of the suture shuttle as the device is closed and opened.

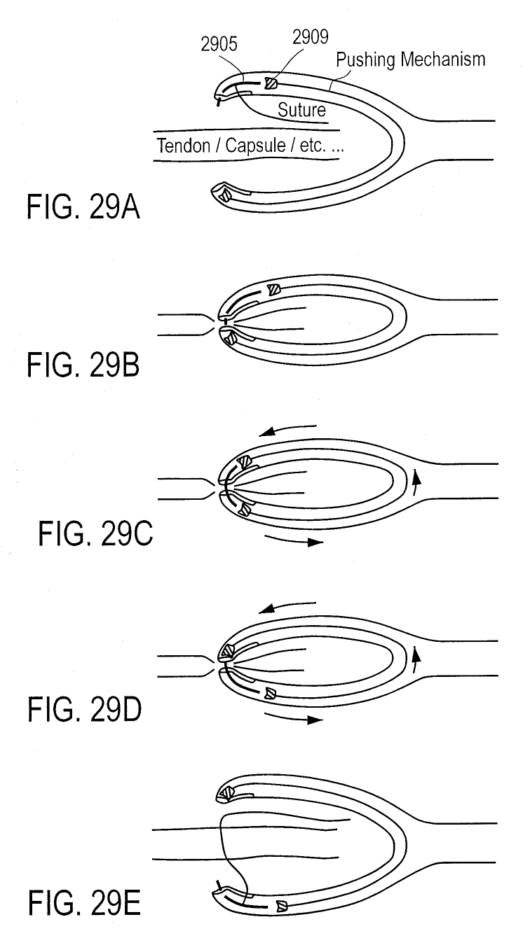

FIGS. 29A-29E illustrate another variation of a suture passer and tissue-penetrating suture shuttle, in which the tissue penetrating suture shuttle may be retracted completely (or at least partially) within the jaws of the suture passer. As mentioned briefly above, retracting the tissue penetrator (including a tissue penetrating suture shuttle) into the arms of the device may protect the suture shuttle from catching on tissue, or otherwise interfering with the operation of the device.

In this variation, the suture shuttle 2905 may be a tissue piercing suture shuttle. The suture shuttle may be held in a suture shuttle dock in either the upper or lower jaw. The suture shuttle dock includes a lock to secure the suture shuttle in the shuttle dock, and also a pusher 2909 to push or pull the suture shuttle that is held in the shuttle dock. In FIGS. 29A-29E this pusher is shown as a reciprocal pusher 2909 so that as the shuttle dock in one jaw is pushed, the opposite shuttle jaw is pulled. In FIGS. 29A-29E the jaws are shown schematically. Any of the jaws (including the parallel opening jaws or the scissor-opening jaws) described previously may be used.

In FIG. 29A the tissue-penetrating suture shuttle 2905 is retracted into the upper jaw of the shuttle dock, and tissue (e.g. a tendon, etc.) is positioned between the jaws. In FIG. 29B the arms of the device are closed over the tissue (slightly compressing the tissue) and the suture shuttle is pushed by the pusher 2909 through the tissue (FIG. 29C), until it engages the shuttle dock in the lower arm (FIG. 29D). Thereafter, the suture shuttle may be retracted at least partially into the shuttle dock of the lower arm, and the arms may be opened to release the tissue.

Another variation of a suture passer that uses a tissue penetrating suture shuttle includes an extendable tissue penetrator that does not itself penetrate the tissue. Instead, the tissue penetrator secures a tissue penetrating suture shuttle at the distal end of the tissue penetrator. The tissue penetrator is extended from the jaw of the suture shuttle when the suture shuttle is held therein, so that it can penetrate the tissue and hand the tissue penetrating suture shuttle to another tissue penetrator on the opposite jaw. Thus, for each pass of the suture shuttle, the tissue penetrator including the tissue-penetrating suture shuttle is extended to meet with the tissue penetrator on the opposite jaw. Thus, in some variations the tissue penetrator does not necessarily include a tissue-penetrating surface (e.g., a sharp surface), but relies on the suture shuttle to penetrate the tissue. Such a tissue penetrator may be referred to as a blunt tissue penetrator or an extendable suture shuttle holder.

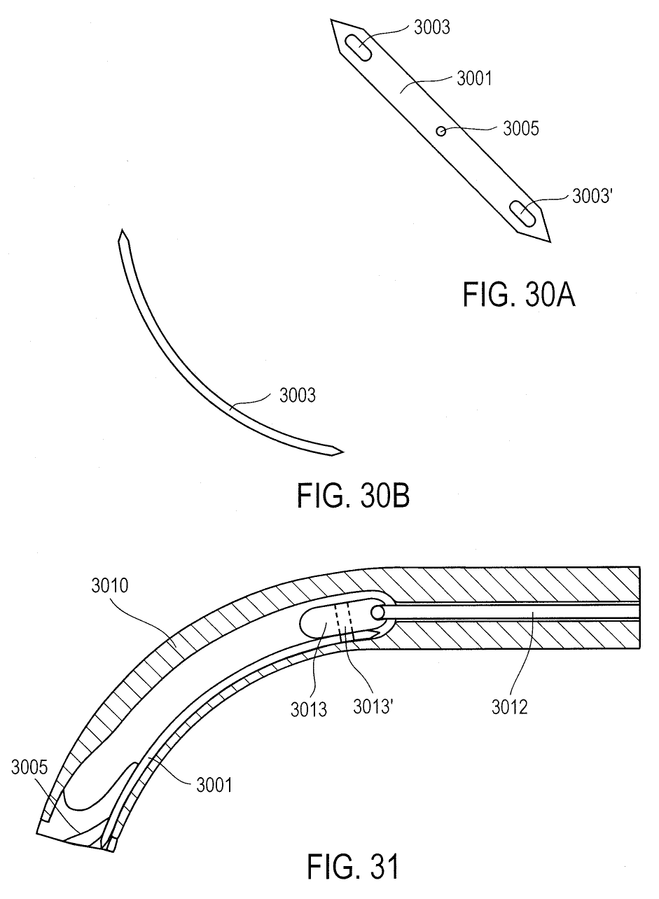

FIGS. 30A and 30B show another variation of a tissue penetrating suture shuttle 3001. The suture shuttle 3001 has tissue-penetrating ends at both ends of the shuttle (in this example, the tissue-penetrating ends are pointed and may be sharp). A suture attachment region 3005 is located in the approximate center of the suture shuttle (shown here as a hole or passage through the suture shuttle). There are openings 3003, 3003′ at near each end of the suture shuttle. These openings may be attachment sites for a shuttle engagement region or lock of a suture passer. For example, the openings may be configured to mate with a pin or other mechanism so that the movement and/or position of the suture shuttle may be controlled by the suture passer. In some variations these openings are pits or indentations. The elongated suture shuttle of FIGS. 30A and 30B may be bent or curved (or configured to bend or curve), as shown in FIG. 30B.

FIG. 31 shows the suture passer of FIGS. 30A and 30B retracted within a jaw of the device. The tissue penetrating suture shuttle 3001 is adjacent to a shuttle dock at the distal end of the jaw 3010; in this example the shuttle dock is a cavity or passage in the distal end of the jaw. The jaw may also include a suture passage (or suture guide) to control the location of the suture when the suture shuttle is located within the jaw. For example, the suture passage may be a channel or opening in the side of the jaw, as previously described. The suture passer with an attached suture may be secured by a shuttle lock 3013 that includes a pin 3013′ or other attachment mechanism. In this example, the attachment mechanism is a retractable pin. The pin maybe spring loaded, so that it extends from the lock body into the opening through the suture shuttle previously mentioned (3003, 3003′ in FIG. 30A, above). The shuttle lock is connected to a wire or pusher 3012 and may be driven in and out of the distal region of the jaw (e.g., the shuttle dock), thereby extending or retracting the tissue penetrating suture shuttle. The distal end of the jaw (the shuttle dock) includes a stopper 3005 that acts as a lock engager or lock disengager. The stopper 3005 is ramped on the same side as the attachment mechanism. As the shuttle lock 3013 is moved into the stopper region 3005, the ramp compresses the spring-loaded stopper 3013′, so that it does not extend from the shuttle lock 3013. If the lock is engaged to a suture shuttle 3001 it this will disengaging the lock from the shuttle, allowing it to be transferred to the shuttle dock in the opposite side. If the shuttle lock 3013 is held within the stopper 3005, as the wire or pusher 3012 withdraws the shuttle lock 3013 from the stopper, the lock will extend from the body of the shuttle lock and may engage the suture shuttle (e.g., an opening in the suture shuttle 3003, 3003′) as previously described. In some variations this wire or pusher 3012 is linked to the wire or pusher in the opposite jaw (e.g., FIG. 29A) so that the movement of the wire or pusher in each jaw is complimentary (pulling one wire occurs simultaneously with pushing the wire in the opposite jaw). As mentioned above, a wire or pusher may be flexible or stiff, and a wire may be a “push” wire so that the wire has sufficient columnar strength to push as well as pull without substantially buckling. In some variations the pusher is a rod.

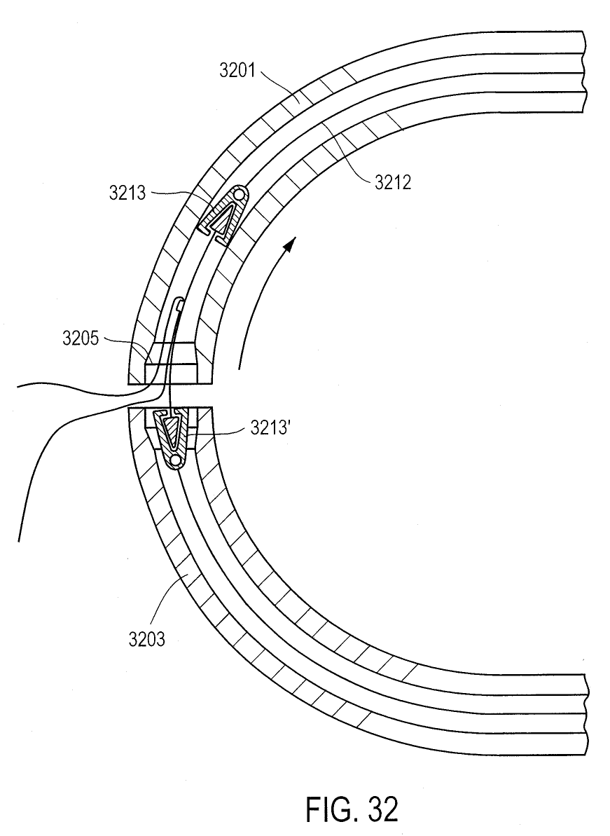

FIG. 32 is another example of a suture passer that may use a tissue-penetrating suture shuttle such as the one shown in FIGS. 30A and 30B. As in FIG. 31, the shuttle lock of FIG. 32 is attached to a wire or pusher 3212. In the variation shown in FIG. 32 the shuttle lock is a flexible collet 3213, 3213′ that has arms that are biased to expand open if the inner diameter of the shuttle dock region is large enough to permit the collet to expand open. For example, the distal end of each shuttle dock region 3205 of the jaws 3201, 3203 is sufficiently large to allow the collet 3213′ to expand open when the collet is positioned at the distal end of the shuttle dock. When the collet is opened it does not engage the suture shuttle, however when the collet is compressed closed (e.g., the upper jaw collet 3213 in FIG. 32), the collet may secure the suture passer so that it can be extended or retracted. The jaws 3201, 3203 of the suture passer in FIG. 32 are shown closed, as they might be when tissue is present between the jaws so that the suture shuttle may be passed between the jaws.

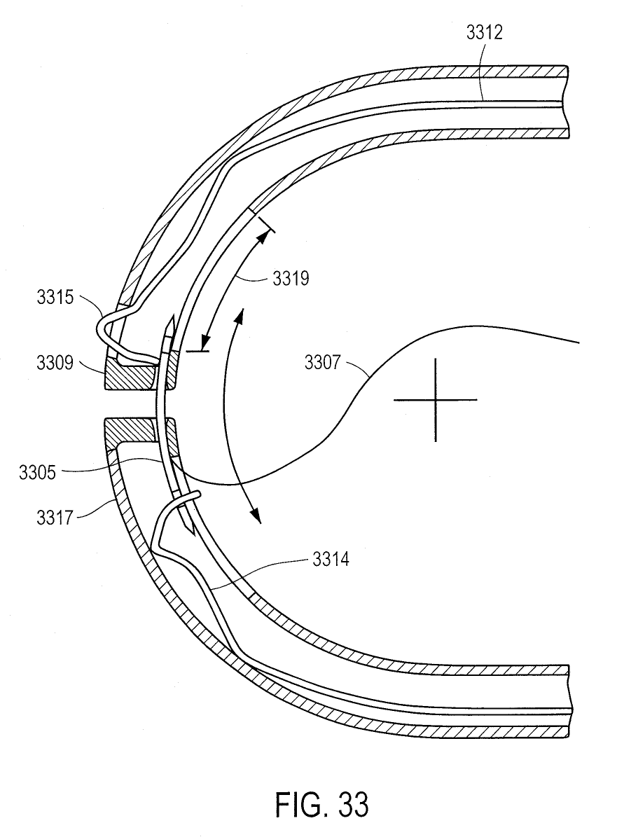

FIG. 33 shows another variation of a suture passer configured to use a tissue penetrating suture shuttle 3305. In this example, the shuttle lock comprises a flexible wire 3314 having a hinged pin 3315 at the end. The end of the shuttle lock can engage the suture shuttle 3305 (e.g., an opening in the suture shuttle 3305, as shown in FIG. 30A) when the shuttle lock wire is compressed within the jaw. The distal end of the jaw includes an opening 3317 into which the shuttle lock may expand, allowing the distal end of the shuttle lock (the tip of the wire) to withdraw from the suture shuttle, as shown in the upper jaw. When the suture shuttle is moved (e.g., by the wire or pusher) further into the upper jaw, the shuttle lock can be compressed back down so that it engage the suture shuttle. When the shuttle lock 3314 is retracted into the jaw, the tip of the shuttle lock is driven back down as the diameter of the walls get smaller, so that the tip can engage the shuttle 3305. As in FIG. 32, the wire or pusher 3312 connected to (or forming) the shuttle lock 3314 in each jaw may be functionally connected, so that as the shuttle lock is withdrawn in one jaw it is extend in the other, and vice-versa. The distal tip of each jaw may be capped by a stopper 3309 that limits the motion of the shuttle lock, preventing it from extending past the distal end of the jaw.

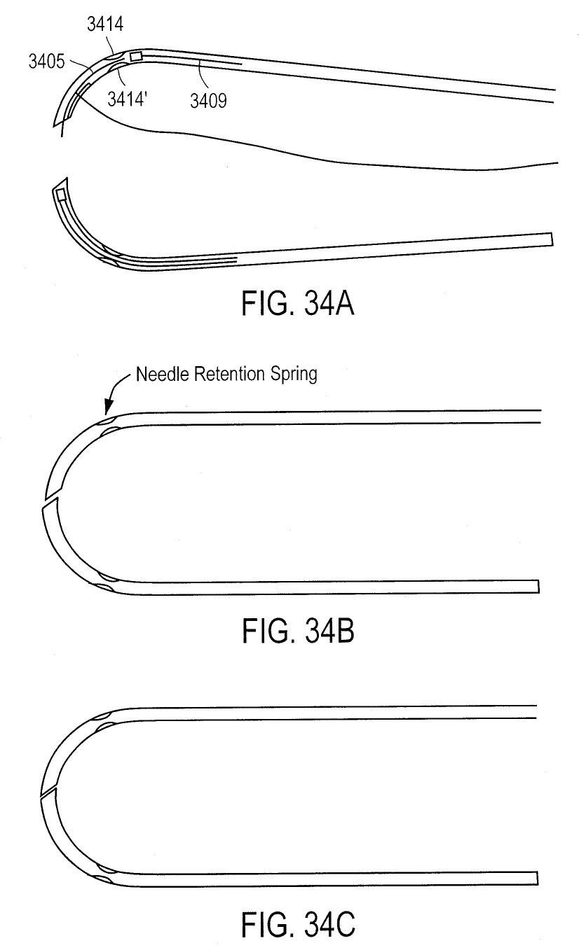

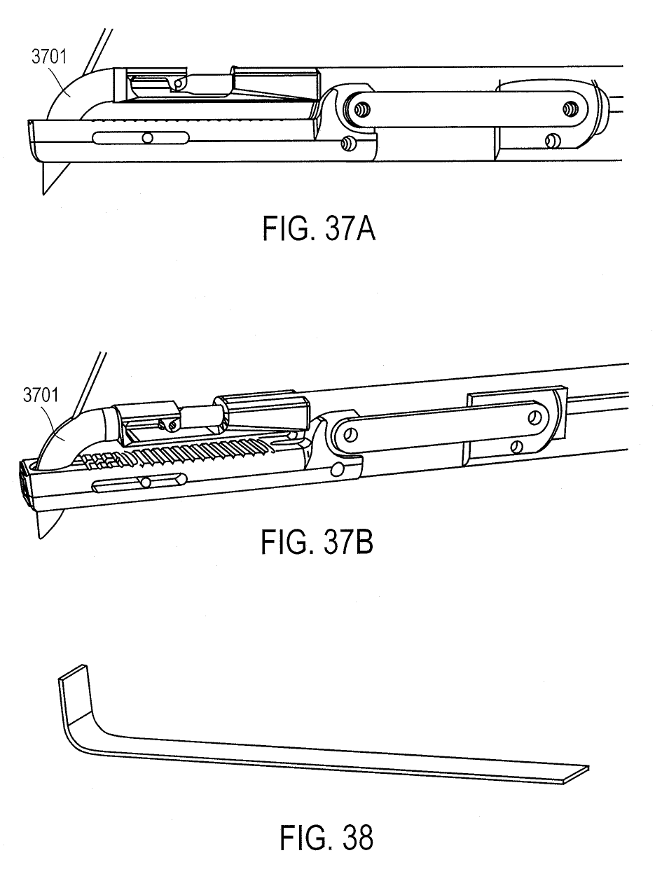

FIGS. 34A-34C describes another variation of a suture passer in which the jaws of the suture passer are tissue penetrating jaws, and the suture shuttle is passed between them. In this variation the suture shuttle may be a blunt suture shuttle 3405. The device illustrated in FIGS. 34A-34C is similar to the suture passer of FIG. 29A-29E in that the suture shuttle may be pushed/pulled from a shuttle dock in each jaw. A suture shuttle pusher (or wire) 3409 may be included. In FIGS. 34A-34C the proximal end of the shuttle dock includes a pair of springs 3411, 3411′ that act as a stopper to help retain the suture shuttle. However, in this example, the jaws are sharp (e.g., pointed, serrated, etc.). For example, after the jaws are opened, and tissue is positioned between them, the jaws may be closed so that the tissue between them is penetrated by the jaws, and the suture shuttle may be passed. FIG. 34B illustrates the jaws in the closed position, forming a complete passage through the tissue for the suture shuttle. In some variations, the jaws are configured to mate with each other in the closed position. In some variations, the jaws are tubular or cannicular. In some variations the jaws are curved, as shown in FIGS. 34A-34C. In some variations only one jaw is tissue-penetrating, as described below in FIGS. 37A-37B.

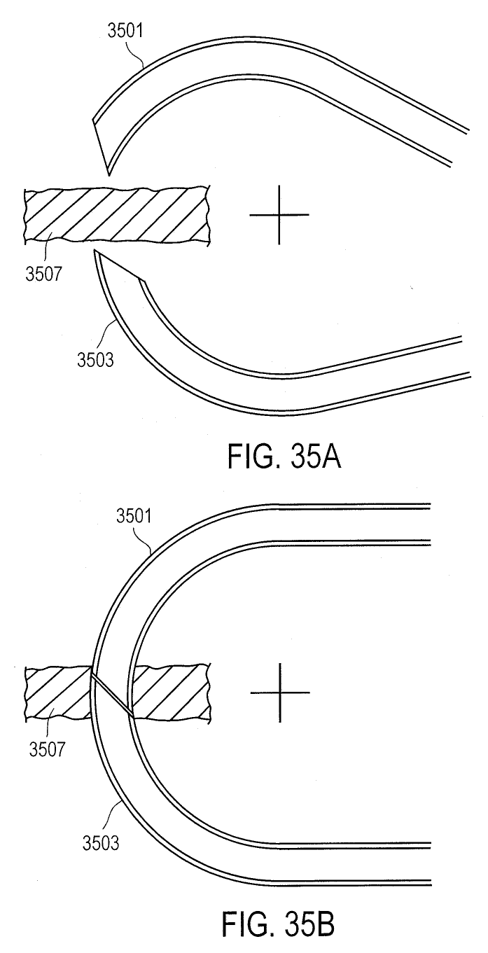

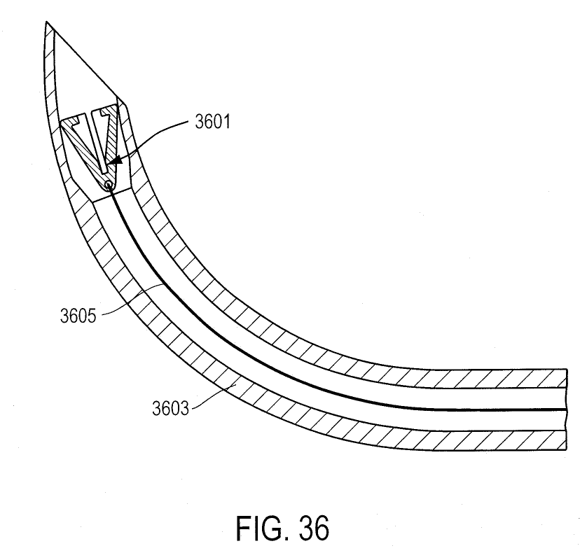

As mentioned above, a suture passer may also be configured so that one or both of the jaws are tissue penetrating. In this variation, a separate extendable tissue penetrator is not necessary. For example, FIGS. 35A and 35B show a variation of a suture passer in which the distal ends of the jaws are tissue-penetrating and each jaw includes a shuttle dock therein so that a suture shuttle may be passed between the jaws. In FIG. 35A a piece of tissue 3507 is positioned between the tissue penetrating jaws 3501, 3503. A suture shuttle (not shown) may be held within either of the jaws shuttle docks so that it may be transferred between the jaws. For example, after the jaws have been closed through the tissue, as shown in FIG. 35B, the suture shuttle may be transferred from one jaw to the other. The shuttle may be transferred by any appropriate method, including those described above. Thus, a pusher or wire may be included in each shuttle dock to extend or retract the suture shuttle so that it can be transferred. The shuttle dock may also include a shuttle lock for securing the shuttle in the shuttle dock. FIG. 36 shows an example of a tissue penetrating jaw 3603 that includes a shuttle lock 3601 and a pusher 3605 that may be used. By coordinating the locking and unlocking of the shuttle within the shuttle dock, as well as the motion of the wire or pusher, the suture shuttle may be passed between the jaws. The variation shown in FIGS. 35A and 35B may also include any of the suture control features (e.g., suture channels or guides) described above. For example, each jaw may include a cut-out region in the side of the jaw at the distal end of each jaw, into which the suture may be positioned as it is transferred from jaw to jaw.

In addition, the tissue penetrating jaws of the variation shown in FIGS. 35A and 35B may engage with each other after they have penetrated the tissue, as shown in FIG. 35B. Thus, the jaws may be aligned so that they form a channel though which the suture shuttle may pass.

FIG. 37A-37B are side and perspective views, respectively of a variation of a suture passing device having a single tissue-penetrating arm. This variation is similar to the example shown in FIG. 4A-C. The tissue-penetrating arm is configured as a tissue penetrator that can hold and pass the suture shuttle (particularly dull or non-tissue penetrating suture shuttles). In this example, the upper jaw is a tissue penetrator 3701. The upper and lower arms are kinematically linked, so that the tissue penetrator will mate with the shuttle dock on the lower jaw as the upper and lower jaws are opened and closed. In some variations, the tissue penetrating upper jaw 3701 is configured to extend upwards, as shown in FIG. 4B, so as to allow unimpeded access to the opening between the jaws. In the variation illustrated in FIGS. 37A and 37B the sharp distal tip of the tissue penetrating upper jaw passes completely through the lower jaw, as previously described for the tissue penetrator (e.g., FIG. 13C). In any of the suture passer variations described herein, the suture passer may include a shield or protector, as shown in FIG. 32. This shield may mount under the opposite jaw to prevent the tissue penetrator from damaging tissue below the opposite jaw.

Any of the devices described herein may be part of a system or kit for passing a suture. For example, any of the suture passers described herein may be included with a suture shuttle as a system for suturing tissue. In some variations, the suture shuttle may be loaded with a suture (e.g. preloaded), or it may be unloaded. For example, the suture shuttle may be threaded with a sterile surgical-grade suture. A kit for suturing may include any of the suture passers described herein, as well as a suture shuttle, and a suture. In some variations the kit may also include instructions for use, including instructions for operating the suture passer, and/or instructions for loading a suture shuttle, and/or instructions for suturing with the suture passer, and/or instructions for knotting or terminating a suture using the device.

Any of the suture passers described herein may be used to suture tissue. In some variations, the suture passer may be used to repair a tissue arthroscopically or endoscopically. For example, the suture passer may be closed and passed through an introducer (e.g., a cannula) into the tissue. In some variations, the suture passer also includes a visualization method. For example, the suture passer may include a channel through which a light source (e.g., fiber optic) or power supply (to power an LED, camera, etc.) may pass. In some variations, the channel may be used to apply or remove fluid or gas.

The devices, systems, kits, and methods described herein may be used to repair any appropriate type of tissue. For example, the devices described herein may be used during arthroscopic rotator cuff repair, open or mini-open rotator cuff repair, arthroscopic labral repair (e.g., Bankart repair or anterior-inferior labral repair, SLAP or superior labrum anterior posterior repair, hip labral repair, etc.), arthroscopic biceps tenodesis, arthroscopic capsular plication, rotator interval closure, capsular shift, arthroscopic capsular repair or reconstruction, arthroscopic meniscus repair or reconstruction, open tendon, ligament and muscle suturing, Achilles tendon repair, ACL repair, or the like. In general the devices described herein may be used for general suturing (laparoscopic, endoscopic, thoracoscopic, transoral, open, cutaneous, etc. Examples include: laparoscopic Nissen fundoplication, laparoscopic Rou-en-Y gastric bypass, laparoscopic Herniorrhaphy, laparoscopic Hiatal Hernia Repair, laparoscopic suturing of the uterus, hemorrhoidectomy, thoracoscopic esophagectomy, intrathoracic esophagogastric anastamosis, transvaginal sacrospinous colpopexy, vaginal prolaps, incontinence procedures, bladder neck suspensions, laparoscopic dismembered pyeloplasties, fistula tract closure, and the like.

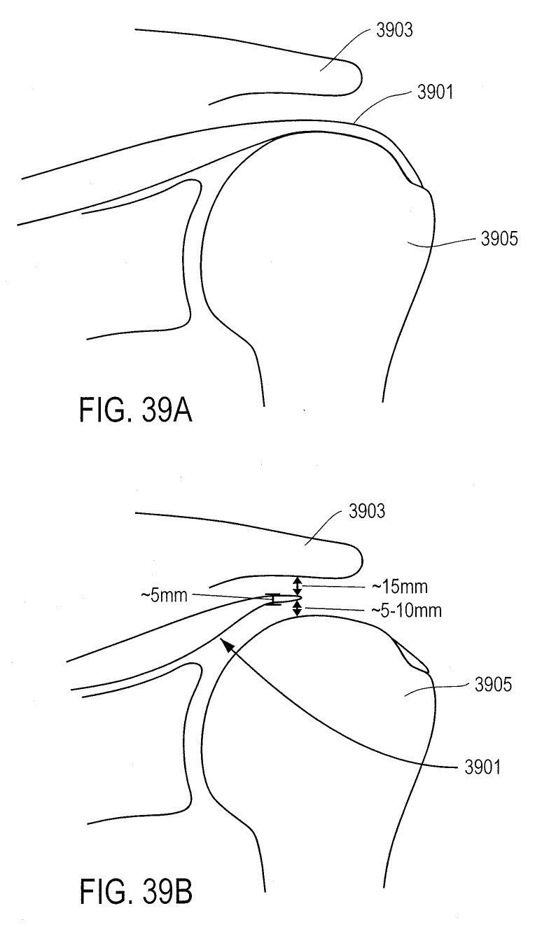

For example, the devices described herein may be used to repair a tissue such as a tendon of the rotator cuff. FIG. 39 is a schematic illustration of a rotator cuff tendon, showing the dimensions of the space into which the suture passer may fit. If the tendon 3901 is torn, a suture passer may fit in the space between the bones 3903 and 3905 to access the tendon, and apply stitches. FIG. 39A shows a tendon in the correct position, when the tendon has been torn, it may be even more difficult to access, as shown in FIG. 39B. There is approximately 25-30 mm of space to work in after traction has been applied to the arm. Two-thirds of this space is above the rotator cuff tendon 3901, and the other third is below. This may mean approximately 15 mm of space above the tendon 3901 and 5-10 mm of space below the tendon (above the head of the bone 3905), assuming the tendon is approximately 5 mm thick.

This region has been traditionally very difficult to access, and to repair. Even if the area is accessed, the practitioner (e.g., surgeon or other medical expert) has had to use a simple or horizontal mattress-type suture stitch, because of the difficulty in access and reliable passing of the suture or a needle holding the suture. However, a high failure rate has been associated with this type of arthroscopic repair of the rotator tendon cuff. It may be desirable to use other types of stitches, such as a modified Mason-Allen stitch, that has been shown to have superior strength when compared with simple and horizontal mattress stitches. The modified Mason-Allen stitch has been commonly used in open rotator cuff repairs, but the easier-to-perform simple and horizontal stitches have been commonly used in arthroscopic rotator cuff repairs because of the technical difficulty of placing the modified Mason-Allen stitch arthroscopically. The continuous suture passers described herein may be readily used to perform a modified Mason-Allen stitch by placing the tissue between the jaws of the device, closing the jaws of the device, passing the suture shuttle through the tissue and between the jaws of the device, and repeatedly repositioning the tissue within the jaws of the device and passing the suture shuttle between the repositioned jaws. The modified Mason-Allen stitch involves placing a basic mattress stitch followed by placing one additional pass beyond the depth of and perpendicular to the mattress stitch. FIGS. 25A-25E is a depiction of a modified Mason-Allen stitch).

In operation, the user (e.g., surgeon) inserts the suture passer through a cannula and positions it across the tissue. Generally, the suture passer may be preloaded with a suture shuttle. The suture shuttle may have a suture attached, or it may be attached to a suture that has already been positioned in the tissue. Once the suture passer is near the tissue to be sutured, the device is opened (e.g., the jaws are retracted) so that tissue may be positioned between the jaws. For example, the user may use the jaw control to open the jaws. For example, a finger trigger may be used to open the jaws. Continuous pressure may be applied to the control to open or close the device. Once the device is positioned, the tissue penetrator may be triggered to penetrate the tissue and form a passage for the suture shuttle. For example, the user may push a Thumb trigger to deploy the suture shuttle through the tissue. The tissue penetrator may latch or engage the opposite jaw. When the suture shuttle is initially in the second jaw, the suture shuttle disengages from the shuttle dock in the second jaw and is engaged to the shuttle dock on the tissue penetrator of the first jaw. The tissue penetrator may then be retracted, pulling a suture attached to the suture shuttle with the suture shuttle through the tissue. The jaw may then be opened and the tissue repositioned between the jaws, and the entire procedure repeated to again pass the suture through the tissue.

In one variation, the suture passer has two jaws and a tissue penetrator retractably connected to the first jaw. The handle of the device includes an upper trigger that is the jaw control, which controls grasping of the jaws independent of a lower control that deploys the tissue penetrator. In this variation, the tissue can be grasped as hard as desired, and the tissue penetrator can be passed at any time. When the tissue penetrator (configured as a hollow needle that includes a shuttle engagement region) hits the lower jaw, the pressure in the second trigger (the tissue penetrator control) increases, which opens the suture shuttle lock (a lever that holds the suture shuttle). This lever operates in an alternating fashion, so that every other time it is activated it will let the shuttle free from the lower jaw. Thus, the suture shuttle is passed between the upper (first) and lower (second) jaws.

Any of the variations of the shuttle passers described herein may also include suture guides, channels or controls to direct the suture as it is passed through the tissue. The suture channels may be open or closed, and may be cavities or channels that are formed within the jaws, tissue penetrator(s) and intermediate regions of the device. The channels may be coated or formed to reduce friction or regions that the suture may catch or tangle on. Control of the suture may be important to the working of any of the devices described herein.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A continuous suture passer comprising:
a first jaw and a second jaw;
a handle including a jaw control to manipulate at least one jaw;
a tissue penetrator configured to form a channel through the tissue through which a suture shuttle may be passed, wherein the suture shuttle may be releasably secured to the tissue penetrator; and
a suture shuttle dock functionally connected to the second jaw and configured to releasably secure the suture shuttle so that the suture shuttle may be passed between the first and second jaws.

2. The device of claim 1, wherein the tissue penetrator is configured to be substantially retractable into the first jaw.

3. The device of claim 2, wherein the distal tip of the tissue penetrator is configured to be retracted into the first jaw.

4. The device of claim 1, further comprising a second tissue penetrator configured to be substantially retractable into the second jaw.

5. The device of claim 1, further comprising a second tissue penetrator configured to be substantially retractable into the second jaw, wherein the second tissue penetrator includes the suture shuttle dock.

6. The device of claim 1, wherein the tissue penetrator further comprises a shuttle engagement region configured to releasably secure the suture shuttle.

7. The device of claim 1, wherein the first jaw comprises a first tissue contacting surface and the second jaw comprises a second tissue contacting surface, and further wherein the first tissue contacting surface and the second tissue contacting surface are substantially parallel when the jaws are open.

8. The device of claim 1, wherein the first jaw and the second jaw are hinged to open in a scissor-like fashion.

9. The device of claim 1, wherein the device is configured so that the relative positions of the tissue penetrator and the suture shuttle dock are substantially the same as the jaws of the device are opened and closed.

10. The device of claim 1, wherein the shuttle dock comprises a latch configured to releasably secure the suture shuttle.

11. The device of claim 1, wherein the first and second jaws comprise the distal end of the device, and further wherein the diameter of the distal end of the device is less than about 15 mm.

12. The device of claim 1, wherein the first and second jaws are less than about 30 mm long.

13. The device of claim 1, further comprising an elongate neck region between the distally located first and second jaws and the proximally located handle.

14. The device of claim 13, wherein the elongate neck region has a diameter that is less than the diameter of the closed first and second jaws.

15. The device of claim 1, wherein the suture shuttle dock is configured to interface with the distal tip of the tissue penetrator.

16. The device of claim 1, wherein the handle further comprises a distal tip deflector control configured to control deflection of the distal end of the device.

17. The device of claim 1, wherein the jaw control comprises a plurality of coaxial members controlling jaw opening and closing.

18. The device of claim 1, wherein the jaw control includes a trigger to which pressure may be applied to control jaw closing.

19. The device of claim 1, wherein the jaw control includes a trigger to which pressure may be applied to control jaw closing and also to deploy the tissue penetrator.

20. The device of claim 1, wherein the jaw control includes a limiter that limits the maximum pressure that the jaws may apply to tissue held between the jaws.

21. The device of claim 1, wherein the handle further comprises a tissue penetrator control configured to extend the tissue penetrator from a retracted position in the first jaw.

22. The device of claim 1, wherein the second jaw comprises a channel through which the tissue penetrator may extend.

23. The device of claim 1, wherein the first and second jaws comprise a substantially transparent material.

24. The device of claim 1, wherein the tissue penetrator comprises a suture passage to at least partially guide the suture when the suture shuttle is engaged with the tissue penetrator.

25. A continuous suture passer comprising:
a first jaw and a second jaw;
a handle including a jaw control to manipulate at least one jaw;
a tissue penetrator configured to form a channel through the tissue through which a suture shuttle may be passed, wherein the distal tip of the tissue penetrator is configured to be substantially retracted into the first jaw; and
a suture shuttle dock functionally connected to the second jaw and configured to releasably secure the suture shuttle so that the suture shuttle may be passed back and forth between the first and second jaws.

26. A continuous suture passer comprising:
a tissue clamping region including
a first jaw having first tissue-contacting surface, and
a second jaw having a second tissue-contacting surface, wherein
the tissue-contacting surfaces of the first and second jaws are substantially parallel when the tissue clamping region is opened;
a handle including a jaw control to manipulate at least one jaw;
an elongate neck region between the distally located tissue clamping region and the proximally located handle; and
a tissue penetrator configured to releasably secure to a suture shuttle and to form a channel through the tissue through which the suture shuttle may be passed.

27. The device of claim 26, wherein the tissue penetrator is configured to be substantially retracted into the first jaw.

28. The device of claim 26, further comprising a suture shuttle dock functionally connected to the second jaw and configured to releasably secure the suture shuttle.

29. The device of claim 28, wherein the relative positions of the tissue penetrator and the suture shuttle dock are substantially the same as the jaws of the device are opened and closed.

30. The device of claim 28, wherein the shuttle dock comprises a latch configured to releasably secure the suture shuttle.

31. The device of claim 26, wherein the tissue penetrator further comprises a shuttle engagement region configured to releasably secure the suture shuttle.

32. The device of claim 31, wherein the shuttle engagement regions comprises a friction fit, a magnetic fit, a snap fit, a pressure fit, or some combination thereof.

33. The device of claim 26, wherein the diameter of the tissue clamping region is less than about 15 mm when closed without any tissue therebetween.

34. The device of claim 22, wherein the tissue clamping region is less than about 30 mm long.

35. The device of claim 26, wherein the elongate neck region has a diameter that is less than the diameter of the closed tissue clamping region.

36. The device of claim 26, wherein the handle further comprises a distal tip deflector control configured to control deflection of the distal end of the device.

37. The device of claim 26, wherein the jaw control comprises a plurality of coaxial members controlling jaw opening and closing.

38. The device of claim 26, wherein the jaw control includes a trigger to which pressure may be applied to control jaw closing.

39. The device of claim 26, wherein the jaw control includes a trigger to which pressure may be applied to control jaw closing and also to deploy the tissue penetrator.

40. The device of claim 26, wherein the jaw control includes a limiter that limits the maximum pressure that the jaws may apply to tissue held between the jaws.

41. The device of claim 26, wherein the handle further comprises a tissue penetrator control configured to extend the tissue penetrator from a retracted position at least partially within the first jaw.

42. The device of claim 26, wherein the second jaw comprises a channel through which the tissue penetrator may extend.

43. The device of claim 26, wherein the first and second jaws comprise a substantially transparent material.

44. The device of claim 26, wherein the tissue penetrator comprises a suture passage to at least partially guide the suture when the suture shuttle is engaged with the tissue penetrator.

45. The device of claim 26, wherein at least the first jaw includes a suture passage through which the suture may reside when the suture shuttle is engaged with the tissue penetrator and the tissue penetrator is retracted into the first jaw.

46. A kit comprising the suture passers of claim 1 or claim 26, further comprising instructions for use.

47. A continuous suture passer comprising:
a first jaw and a second jaw;
a handle including a jaw control to manipulate at least one jaw;
a first tissue penetrator configured to extend from the first jaw and to form a channel through the tissue through which a suture shuttle may be passed, wherein the suture shuttle may be releasably secured to the first tissue penetrator; and
a second tissue penetrator configured to extend from the second jaw and to form a channel through the tissue which the suture shuttle may be passed, wherein the suture shuttle may be releasably secured to the second tissue penetrator;
wherein the first and second tissue penetrators are configured to pass the suture shuttle between them.

48. A suture shuttle comprising:
a suture attachment region configured to secure to a suture;
a shuttle body having an outer surface that is substantially blunt, wherein the shuttle body region has an outer diameter that is less than 5 mm; and
a shuttle retainer region configured to engage a tissue penetrator to retain tire suture shuttle in communication with the tissue penetrator.

49. The device of claim 48, further comprising a shuttle alignment feature, wherein the shuttle alignment feature communicates with a region of the tissue penetrator and a region of a suture shuttle dock to orient the suture shuttle.

50. The device of claim 49, wherein the shuttle alignment feature comprises a groove in the shuttle body.

51. The device of claim 48 wherein the shuttle body region is substantially spherical, substantially ellipsoid, or substantially elongate.

52. The device of claim 48, wherein the suture attachment region comprises a loop, a hook, a passage, or a channel.

53. The device of claim 48, wherein the shuttle body region comprises a polymer, a metal, or a ceramic.

54. The device of claim 48, wherein the shuttle retainer region comprises a portion of the outer surface of the shuttle body.

55. The device of claim 48, further comprising a suture attached to the suture attachment region of the shuttle.

56. A system for continuously suturing a tissue, the system comprising;
a continuous suture passer having
a first jaw and a second jaw,
a handle including a jaw control configured to manipulate at least one jaw, and
a tissue penetrator configured to form a channel through the tissue, wherein the tissue penetrator is configured to be substantially retractable into the first jaw; and
a suture shuttle having
a suture attachment region configured to secure to the distal portion of a surgical suture, and
a shuttle body that is configured to engage the tissue penetrator.

57. The system of claim 56, further comprising a suture attached to the suture attachment region of the shuttle.

58. The system of claim 56, wherein the continuous suture passer further comprises a suture shuttle dock functionally connected to the second jaw and configured to releasably secure the suture shuttle.

59. The system of claim 56, wherein the continuous suture passer further comprises a tissue clamping region including a first tissue-contacting surface on the first jaw and a second tissue-contacting surface on the second jaw, wherein the tissue-contacting surfaces of the first and second jaws are substantially parallel when the tissue clamping region is opened.

60. A method of passing a suture comprising:
positioning a tissue between a first jaw and a second jaw of a suture passer, wherein the suture passer includes an extendable tissue penetrator functionally connected with the first jaw and a suture shuttle dock functionally connected with the second jaw, wherein a suture shuttle is releasably held by either the suture shuttle dock or the retractable tissue penetrator;
extending the tissue penetrator through the tissue from a retracted position;
transferring the suture shuttle between the suture shuttle dock and the retractable tissue penetrator;
retracting the tissue penetrator through the tissue;
repositioning the tissue between the first jaw and the second jaw of the suture passer;
extending the tissue penetrator through the tissue; and
transferring the suture shuttle between the suture shuttle dock and the retractable tissue penetrator.

61. The method of claim 60, further comprising securing the tissue between the first jaw and the second jaw before extending the tissue penetrator through the tissue.

62. The method of claim 60, further comprising loading a suture shuttle into the suture passer.

63. The method of claim 60, further comprising attaching a suture to the suture shuttle.

64. The method of claim 60, further comprising separating the first and second jaws so that the first and second jaws are substantially parallel.

65. The method of claim 60, wherein the suture shuttle is transferred from the suture shuttle dock to the tissue penetrator by contacting the suture shuttle with the tissue penetrator and releasing the suture shuttle from the suture shuttle dock.

66. The method of claim 60, wherein the suture shuttle is transferred from the tissue penetrator to the suture shuttle dock by disengaging the suture shuttle from the extended tissue penetrator.

67. The method of claim 60, further comprising forming a modified Mason-Allen stitch.

68. The method of claim 60, wherein the tissue comprises rotator cuff tendon.

69. A method of passing a suture comprising:
expanding a tissue clamping region of a suture passer, wherein the tissue clamping region has a first jaw with a first tissue-contacting surface and a second jaw with a second tissue-contacting surface, so that the first tissue-contacting surface and the second, tissue-contacting surface are substantially parallel;
positioning a tissue between the first and second jaws;
clamping the tissue between the first and second jaws;
extending a retractable tissue penetrator into the tissue from the first jaw after clamping the tissue between the first and second jaws;
engaging a suture shuttle with the tissue penetrator; and
retracting the tissue penetrator and pulling the suture shuttle through the tissue.

70. The method of claim 69, wherein the tissue is clamped between the first and second jaws with a pressure between the first and second jaws that is less than 20 lbs.

71. The method of claim 69 wherein the suture passer further comprises a handle having a jaw control, and the tissue is clamped between the first and second jaws of the suture passer by applying pressure to a jaw control on a handle.

72. The method of claim 71, wherein the jaw control comprises a limiter that limits the amount of pressure applied by the first and second jaws against the tissue when pressure is applied to the jaw control.

73. The method of claim 72, wherein the retractable tissue penetrator is extended when the pressure applied to the jaw control exceeds a predetermined amount.

74. The method of claim 69, further comprising unclamping the tissue from between the first and second jaws.

75. The method of claim 69, further comprising repositioning the tissue between the first and second jaws and then clamping the tissue between the first and second jaws.

76. The method of claim 69, further comprising extending the retractable tissue penetrator and engaged suture shuttle through the tissue.

77. The method of claim 69, wherein the tissue penetrator is extended through the tissue to contact at least a portion of the second jaw.

78. The method of claim 69, wherein the tissue is rotator cuff tendon tissue.

79. The method of claim 69, wherein the tissue is Labral tissue.

80. The method of claim 69, wherein the tissue is capsular tissue.

81. The method of claim 69, further comprising laproscopically inserting the tissue clamping region of a suture passer into a subject.

82. A continuous suture passer comprising:
a first jaw and a second jaw;
a handle including a jaw control to manipulate at least one jaw;
a first suture shuttle dock functionally connected to the first jaw and a second suture shuttle dock functionally connected to the second jaw, wherein the first and second suture shuttle docks are configured to releasably secure a suture shuttle so that the suture shuttle may be passed between the first and second jaws; further wherein the first suture shuttle dock includes a suture passage to at least partially guide a suture connected to the suture shuttle when the suture shuttle is engaged with the first suture shuttle dock.

83. The device of claim 82, further comprising a second suture passage to at least partially guide a suture connected to the suture shuttle when the suture shuttle is engaged with the second suture shuttle dock.

84. The device of claim 82, wherein the suture passage comprises a notch or opening in the shuttle dock through which the suture may be guided.

85. The device of claim 82, wherein the suture passage has a substantially smooth surface against which the suture may rest.

86. The device of claim 82, further comprising an intermediate region between the first and second jaws and the handle, wherein the intermediate region comprises a suture channel configured to guide the suture extending from the first or second jaws.

87. The device of claim 86, wherein the suture channel comprises an opening extending longitudinally along the intermediate region.
